(19) United States
(12) Patent Application Publication
(10) Pub. No.: US 2019/0209697 A1
(43) Pub. Date: Jul. 11, 2019

(54) CELLS LABELLED WITH LIPID CONJUGATES AND METHODS OF USE THEREOF

(71) Applicant: The Regents of the University of California, Oakland, CA (US)

(72) Inventors: Elliot C. Woods, Burlingame, CA (US); Carolyn R. Bertozzi, Stanford, CA (US)

(21) Appl. No.: 15/769,047

(22) PCT Filed: Nov. 3, 2016
(86) PCT No.: PCT/US2016/060298

§ 371 Date: Apr. 17, 2018

Related U.S. Application Data
(60) Provisional application No. 62/251,389, filed on Nov. 5, 2015.

(51) Int. Cl. A61K 47/54; A61K 9/50; A61K 31/715; A61P 37/06

(52) U.S. Cl. CPC A61K 47/554; A61P 37/06; A61K 31/715; A61K 9/5068

(57) ABSTRACT

A method of labelling a cell is provided. Aspects of the method include the contacting the cell with a cholesteryl-cargo conjugate having the formula: C-L-Z (I) wherein C is a cholesterylamine anchor group, L is an optional linker and Z is a linked cargo moiety to non-covalently bind the cholesterylamine anchoring group to the cell membrane thereby displaying Z at the cell surface for an extended period of time. Aspects of the method further include administering the labelled cell to a subject. Also provided is a method of modulating an immune response in a subject and a method of targeting a cell in a subject. Cholesterylamine conjugates, labelled cells and kits including the same that fmd use in the subject methods are also provided.

### CROSS-REFERENCE

This application claims the benefit of U.S. Provisional Patent Application No. 62/251,389, filed Nov. 5, 2015, which application is incorporated herein by reference in its entirety.

### STATEMENT REGARDING FEDERALLY SPONSORED RESEARCH

This invention was made with government support under Grant No. CA200544 and GM059907 awarded by the National Institutes of Health. The government has certain rights in the invention.

### INTRODUCTION

Global changes in glycosylation can accompany inflammation, microbial infection and tumorigenesis. For example, heavily glycosylated mucin glycoproteins are known to be upregulated in epithelial carcinomas. Mucins are membrane-associated glycoproteins with densely glycosylated ectodomains; their glycans often comprise >50% of the mass of the molecule. Mucins' biophysical bulk may actually bolster adhesion of tumor cells in minimal adhesion settings, such as the metastatic niche. And broad upregulation of certain glycan motifs, such as sialic acid, can play an important role in how the tumor and the host immune system interact. An increase in cell-surface sialic acid expression can change the polarity of tumor-resident macrophages, block “eat-me” signals to cells responsible for immune editing, and reduce the ability of Natural Killer (NK) cells to destroy transformed cells. Studies of glycobiology have benefitted from chemical approaches, such as the display of synthetic glycoconjugates on live cells via chemical glycocalyx engineering or the covalent attachment of glycoconjugates to engineered cell-surface molecules.

### SUMMARY

A method of labelling a cell is provided. Aspects of the method include contacting the cell with a cholesteryl-cargo conjugate having the formula: C-L-Z (I) wherein C is a cholesterylamine anchor group, L is an optional linker and Z is a linked cargo moiety to non-covalently bind the cholesterylamine anchoring group to the cell membrane thereby displaying Z at the cell surface for an extended period of time. Aspects of the method further include administering the labelled cell to a subject. Also provided is a method of modulating an immune response in a subject and a method of targeting a cell in a subject. Cholesterylamine conjugates, labelled cells and kits including the same that find use in the subject methods are also provided.

The present disclosure provides a method of labelling a cell, the method comprising: contacting the cell with a cholesteryl-cargo conjugate having the formula: C-L-Z (I), wherein: C is a cholesterylamine anchor group; L is an optional linker; and Z is a linked cargo moiety; to non-covalently bind the cholesterylamine anchoring group to the cell membrane thereby displaying Z at the cell surface for an extended period of time. In some cases, the extended period of time is 1 day or more (e.g., 2 days, 3 days, 4 days, 5 days, 6 days, 7 days, from 1 week to 2 weeks, or more than 2 weeks). In some cases, 2% or more (e.g., fro 2% to 5%, from 5% to 10%, from 10% to 25%, from 25% to 50%, or more than 50%) by molarity of the cargo moiety taken up by the cell is displayed at the cell surface. In some cases, the cholesteryl-cargo conjugate is recycled to and from the cell surface via internalized vesicles that non-covalently bind the cholesterylamine anchor group. In some cases, the linked cargo moiety is selected from an immunoinhibiting agent, an immunoactivating agent, an immunomodulating agent, an adhesion modulating agent, a cell surface displayed therapeutic agent, a targeting agent, a cell differentiation agent and an imaging agent. In some cases, the method further comprises cleaving the linked cargo moiety from the cholesterylamine anchor group. In some cases, the cell is selected from an immune cell, a stem cell, an autologous cell, an allographic cell, xenographic cell and a host cell for expression of a target. In some cases, the method further comprises administering the labelled cell to a subject. In some cases, the linked cargo moiety is a cell surface displayed therapeutic agent. In some cases, the linked cargo moiety is an imaging agent selected from a fluorescent probe, an IR dye, a PET emitting probe and a NMR contrast agent. In some cases, the linked cargo moiety is a cell differentiation agent. In some cases, the wherein the linked cargo moiety is selected from a glycan, a glycopolymer, a protein, a small molecule and a peptide.

The present disclosure provides a method of modulating an immune response in a subject, the method comprising: administering to a subject a labelled cell comprising: a cell; and a cholesteryl-cargo conjugate having the formula: C-L-Z (I), wherein: C is a cholesterylamine anchor group bound to the cell membrane; L is an optional linker; and Z is a linked cargo moiety displayed at the cell surface and selected from an immuno-inhibiting agent, an immuno-activating agent and an immuno-modulating agent; to modulate an immune response of the subject. In some cases, the linked cargo moiety is an immuno-inhibiting agent; and In some cases, the cell is a transplanted cell. In some cases, the linked cargo moiety is an immuno-activating agent; and the cell is an autologous cell further comprising a tumor targeting agent. In some cases, the linked cargo moiety is an immuno-modulating agent; and the cell is an immune system cell.

The present disclosure provides a method comprising: administering to a subject a labelled cell comprising: a cell; and a cholesteryl-cargo conjugate having the formula: C-L-Z (I), wherein: C is a cholesterylamine anchor group bound to the cell membrane; L is an optional linker; and Z is a linked cargo moiety displayed at the cell surface and is a cell adhesion modulating agent or a targeting agent; to locally target the cell or to modulate adhesion of the cell to an extracellular matrix of the subject. In some cases, the linked cargo moiety is an adhesion-promoting agent; and the cell is a stem cell. In some cases, the linked cargo moiety is an adhesion-inhibiting agent; and the cell is a therapeutic agent expressing cell. In some cases, the linked cargo moiety is a targeting agent.

The present disclosure provides a cholesterylamine conjugate having the formula: C-L-Z wherein: C is an amine-linked cholesterylamine; L is an optional linker; and Z is selected from an immunoinhibiting agent, an immunoactivating agent, an immunomodulating agent, an adhesion modulating agent, a cell surface displayed therapeutic agent, a targeting agent, a cell differentiation agent and an imaging agent. In some cases, Z comprises a glycan selected from Sia, N-acetylneuraminic acid; lactose, galactose-b1,4-glucose; GalNAc, N-acetylgalactosamine; GD3, N-acetylneuraminic acid-α2,8-N-acetylneuraminic acid-α2,3-galactose-β1,4-glucose; and SiaLex, N-acetylneuraminic acid-α2,3-galactose-β1,4-(α1,3-fucose)-N-acetylglucosamine. In some cases, Z has the following structure:

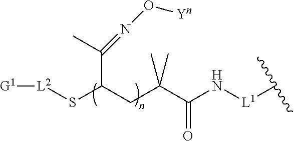

wherein: each Y is independently a glycan or sidechain group; L¹ and L² are each optional linkers; n is an integer from 1 to 10,000; and G¹ is selected from H, an alkyl, a substituted alkyl and a detectable moiety.

The present disclosure provides a labelled cell, the cell comprising: a cell; and a cholesterylamine-cargo conjugate having the formula: C-L-Z (I), wherein: C is a cholesterylamine anchor group bound to the cell membrane; L is an optional linker; and Z is a linked cargo moiety displayed at the cell surface, wherein Z is selected from an immunoinhibiting agent, an immunoactivating agent, an immunomodulating agent, an adhesion modulating agent, a cell surface displayed therapeutic agent, a targeting agent, a cell differentiation agent and an imaging agent. In some cases, the cell is an immune cell. In some cases, the cell is a tumor cell. In some cases, the cargo moiety is a glycopolymer. In some cases, the cell comprises a plurality of cholesterylamine-cargo conjugates displaying a plurality of linked cargo moieties at the cell surface. In some cases, the plurality of linked cargo moieties defines a glycocalyx layer at the surface of the labelled cell. In some cases, the cell is selected from a stem cell, a transplanted allographic cell, an autographic cell, a xenographic cell, a bacterial cell and an immune cell. In some cases, the cell is and in vitro cell. In some cases, the cell is an ex vivo cell. In some cases, the cell is an in vivo cell. In some cases, the cell further comprises a second cholesterylamine-cargo conjugate comprising a second distinct linked cargo moiety that is located at the cell surface. In some cases, the cholesteryl-anchored tag has the structure:

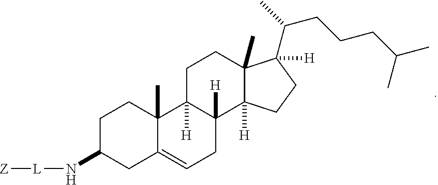

In some cases, Z is a glycopolymeric mimic of a mucin. In some cases, Z has the following structure:

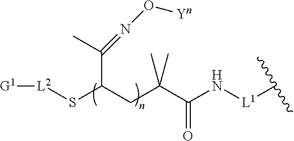

wherein: each Y is independently a glycan or a sidechain group; L¹ and L² are each optional linkers; n is an integer from 1 to 10,000; and G¹ selected from H, an alkyl, a substituted alkyl and a detectable moiety. In some cases, each Y is selected from Sia, N-acetylneuraminic acid; lactose, galactose-b1,4-glucose; GalNAc, N-acetylgalactosamine; GD3, N-acetylneuraminic acid-α2,8-N-acetylneuraminic acid-α2,3-galactose-β1,4-glucose; SiaLex, N-acetylneuraminic acid-α2,3-galactose-β1,4-(α1,3-fucose)-N-acetylglucosamine, acetic acid and glycerol. In some cases, Z has the following structure:

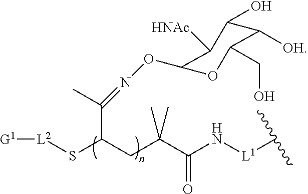

The present disclosure provides a kit for labelling a cell, the kit comprising: a cholesterylamine conjugate or precursor thereof having the formula: C-L-Z (I), wherein: C is a cholesterylamine anchor group; L is an optional linker; and Z is a chemoselective reactive group or a linked cargo moiety; and at least one component selected from a cell, a cargo moiety, and a positive or negative control.

### BRIEF DESCRIPTION OF THE DRAWINGS

The invention is best understood from the following detailed description when read in conjunction with the accompanying drawings. The patent or application file contains at least one drawing executed in color. It is emphasized that, according to common practice, the various features of the drawings are not to-scale. On the contrary, the dimensions of the various features are arbitrarily expanded or reduced for clarity. Included in the drawings are the following figures. It is understood that the drawings, described below, are for illustration purposes only. The drawings are not intended to limit the scope of the present teachings in any way.

FIG. 1 shows a schematic of cholesterylamine-anchored glycopolymers that persist on cell membranes. The lipid constructs are internalized but recycle to allow for the continuous display of cell-surface glycopolymers for an extended period of time, e.g., up to 10 days.

FIG. 2 illustrates the structure of exemplary cholesterylamine-anchored glycopolymers. A panel of alkynyl lipids that can be clicked to an azide-functionalized RAFT agent were developed (2) and become the anchor for the polymer, R.

FIG. 3 depicts a scheme for synthesis of lipid-anchored dual-end-functionalized glycopolymers. To the known azide-functionalized chain transfer agent (10), an alkynyl lipid (R) is added. The resulting lipid-functionalized CTA (11) is used to polymerize MVK to give the polymers (12). The trithiocarbonate is cleaved and the resulting free-thiol terminated polymers (13) are reacted with maleimide-probe (e.g., R² is biotin or AF488). To the dual functionalized poly-MVK polymer (14), aminooxy GalNAc is added, resulting in a probe-capped lipid-anchored glycopolymer (15).

FIG. 4, panels A and B, illustrates that glycopolymers with different lipid anchors have different cell-surface kinetics. Panel A): The efficiency of cell labeling is highly dependent on lipid chemistry. The shortest phosphocholines and the sterol lipids have the highest labeling efficiencies. Panel B): Relative fractional cell-surface retention of biotin-capped polymers as measured by labeling with an anti-biotin antibody at the given time point and analyzing by flow cytometry. None of the lipids in the panel seem to robustly resist uptake by the cell.

FIG. 5, panels A and B, illustrates cell-surface half-life and fluorescence of cells displaying CholA-anchored polymers. Panel A illustrates CholA-anchored polymers display double exponential kinetics on long time scales. An initial cell-surface half-life of 0.25 hours is superseded by one of 39 hours. For comparison, DPPE-anchored biotin-capped polymers are nearly undetectable after 24 hours (single-exponential decay kinetics with t_{1/2}=5.8 hours). Panel B illustrates the fluorescence of cells labeled with a cytosolic cell-tracking dye, which can be used to monitor cell division. Cells proliferate for days with or without CholA-anchored polymers. This suggests that the CholA-anchored polymers are transferred to daughter cells.

FIG. 6, panels A-C, shows that CholA-anchored glycopolymers recycle from reservoirs inside the cell to the cell surface. Panel A): Jurkats were labeled with 5 μM AF488-capped glycopolymers for 1 h at RT, then washed and returned to warm complete media to incubate for the indicated times. After 2 hours at 37° C., CholA polymers can be seen accumulating in dense areas within cells, whereas DPPE polymers don't form such reservoirs. Panel B) shows AF647-labeled human transferrin, a marker for the ERC, colocalizes with the reservoir formed by AF488-capped CholA-anchored glycopolymers, suggesting that recycling contributes to the sustained residence times of the polymer. Panel C) shows AF488-capped glycopolymers at the cell's surface can be quenched with an anti-AF488 antibody. Cells then allowed to incubate for 2 h display a temperature-dependent return of cell-surface labeling, further suggesting that CholA-anchored polymers are recycling from internal reservoirs to the cell surface.

FIG. 7 illustrates a kinetic funnel model for glycocalyx-driven integrin clustering. By hindering the majority of integrin ligands, bulky ectodomains encourage binding events to occur where bonds already exist, forcing integrins to cluster and thus signal the cell to resist apoptosis.

FIG. 8, panels A and B, shows CholA-anchored glycopolymers are excluded from sites of focal adhesion formation and drive a resistance to anchorage-dependent cell death. Panel A) Nonmalignant MCF-10A cells plated on soft polyacrylamide substrates undergo anoikis—anchorage-dependent cell death. Long (90 nm) CholA-anchored glycopolymers prevent anoikis, whereas short (3 nm) polymers or vehicle (PBS), do not, suggesting a biophysical influence of the glycocalyx on integrin signaling. Panel B): Quantification of the Manders' Colocalization Coefficients for coincidence of mCherry-paxillin with AF488-anti-biotin antibody. Long CholA-anchored glycopolymers show significantly less colocalization than short CholA polymers, suggesting a size-dependent exclusion of polymers from focal adhesions.

FIG. 9 shows TIRF imaging of mCherry-paxillin expressing MCF-10A breast epithelial cells incubated with biotin-capped CholA polymers and stained with AF488-anti-biotin. Polymers with large (90 nm) glycodomains are excluded from sites of adhesion, while polymers with short (3 nm) domains are not—evidence for the kinetic trap model of integrin clustering.

FIG. 10, panels A and B, demonstrates long CholA-anchored glycopolymers protect previously nonmalignant cells from adhesion-mediated apoptosis in vivo. Panel A) Zebrafish embryos, 48 hpf, were injected with GFP-H2B expressing MCF-10A cells incubated with either long (90 nm) or short (3 nm) CholA-anchored glycopolymers. Fish were then imaged at 3 hours and again at 27 hours. Cells remaining viable continue to express GFP-H2B, and their nuclei can be quantified. Panel B) shows that cells with long polymers were almost twice as likely to survive to 27 hours compared to cells with short polymers or cells with no polymers.

FIG. 11 illustrates a general synthesis of alkynyl tagged lipids (e.g., phosphocholines).

FIG. 12 illustrates a synthesis of an alkynyl cholesterylamine

FIG. 13 illustrates that lipid-anchored glycopolymers exhibit saturation on cell surfaces.

FIG. 14 shows that exemplary CholA conjugates can be detected and measured on cell surfaces up to ten days after labeling.

FIG. 15 shows that CholA conjugates can be driven by mass action out of the cell surface.

FIG. 16, panels A and B, shows that CholA conjugates are distributed equitably between daughter cells.

FIG. 17 shows that uptake of CholA conjugates is temperature dependent.

FIG. 18, panels A and B, shows that CholA-anchored glycopolymers are still present on cell surfaces after 24 hours.

FIG. 19, panels A and B, shows data demonstrating long CholA polymers (Panel A) and short CholA polymers (Panel B) exhibit long lifetimes.

FIG. 20 shows that CholA conjugates are long-lived on MCF-10As.

FIG. 21 illustrates that long CholA-anchored polymers increase the adhesion size of MCF-10A cells.

FIG. 22 shows images of tails of zebrafish injected with GFP-H2B expressing MCF-10A breast epithelial cells loaded with either long or short CholA-anchored polymers.

### DEFINITIONS

Before describing exemplary embodiments in greater detail, the following definitions are set forth to illustrate and define the meaning and scope of the terms used in the description. Unless defined otherwise, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D D., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

As used here, the terms “lipid conjugate” and “lipid construct” are used interchangeably and refer to a lipid (e.g., a lipid group derived from cholesterylamine) covalently conjugated to a cargo moiety via an optional linker.

“Isolated” or “purified” generally refers to isolation of a substance (e.g., compound, polynucleotide, protein, cell) such that the substance comprises the majority percent of the sample in which it resides. In some cases, in a sample a substantially purified component comprises 50% or more, such as 80%-85% or more, or 90-95% or more of the sample.

As used herein, the terms “linker”, “linkage” or “linking group” refer to a linking moiety that connects two groups. In some instances, the linker may have a backbone of 100 atoms or less in length, e.g., 50 atoms or less in length, including 20 atoms or less in length. A linker may be a covalent bond that connects two groups or a chain of between 1 and 20 atoms in length, for example of about 1, 2, 3, 4, 5, 6, 8, 10, 12, 14, 16, 18 or 20 carbon atoms in length, where the linker may be linear, branched, cyclic or a single atom. In certain cases, one, two, three, four or five or more carbon atoms of a linker backbone may be optionally substituted with a sulfur, nitrogen or oxygen heteroatom. The bonds between backbone atoms may be saturated or unsaturated. In some instances, no more than one, two, or three unsaturated bonds will be present in a linker backbone. The linker may include one or more substituent groups, for example an alkyl, aryl or alkenyl group. A linker may include, without limitations, oligo(ethylene glycol), ethers, thioethers, tertiary amines, amino acid residues, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone.

In certain embodiments, the linking group comprises 1-15 carbon atoms and/or 0-6 heteroatoms.

In certain embodiments, the linking group is selected from the group consisting of —(CH₂)n-C(O)—, or —C(O)(CH₂)n- or —C(O)(CH₂)n-NHC(O)—, or —C(O)(CH2)n-NHC(O)(CH₂)n-, or —(CH₂)nSCH₂C(O)—, or —(CH₂)n-C(O)NH—(CH2)n-, or —(CH₂)n-NH—C(O)—, or —(CH₂)n-NH—C(O)—(CH₂)n-, or —C(O)—(CH₂)n-, or —(CH₂)n-NH—; and n is an integer from 1 to 10, and including acid salts thereof. In certain embodiments, the linking group is —(CH₂)n-C(O)NH—(CH₂)n-, where each n is an integer from one to ten. In certain embodiments, the linking group is —(CH₂)n-C(O)NH—(CH₂)n-, where each n is one or two. In certain embodiments, the linking group is —(CH₂)n-, where n is an integer from one to ten. In certain embodiments, the linking group is —(CH₂)—. In certain embodiments, the linking group is —(CH₂)n-C(O)N(CH₂)n(CH₃)—(CH₂)n-, where each n is an integer from one to ten. In certain embodiments, the linking group is —(CH₂)n-C(O)N(CH₂)n(CH₃)—(CH₂)n-, where each n is one or two. In certain embodiments, the linking group is —(CH₂)n-C(O)N(CH₃)—(CH₂)n-, where each n is an integer from one to ten. In certain embodiments, the linking group is —(CH₂)n-C(O)N(CH₃)—(CH₂)n, where each n is one or two. In certain embodiments, the linking group comprises 10-15 carbon atoms and/or 0-6 heteroatoms. Additionally, linkers can comprise modified or unmodified nucleotides, nucleosides, polymers, sugars and other carbohydrates, polyethers, such as for example, polyethylene glycols, polyalcohols, polypropylenes, propylene glycols, mixtures of ethylene and propylene glycols, polyalkylamines, polyamines such as spermidine, polyesters such as poly(ethyl acrylate), polyphosphodiesters, and alkylenes.

A linker may be cleavable or non-cleavable. As used herein, the term “cleavable linker” refers to a linker that can be selectively cleaved to produce two products. Application of suitable cleavage conditions to a molecule containing a cleavable linker that is cleaved by the cleavage conditions will produce two cleavage products. A cleavable linker may be stable, e.g. to physiological conditions, until it is contacted with a stimulus capable of cleaving the cleavable linker.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

“Alkyl” refers to monovalent saturated aliphatic hydrocarbyl groups having from 1 to 10 carbon atoms and such as 1 to 6 carbon atoms, or 1 to 5, or 1 to 4, or 1 to 3 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C-), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain have been optionally replaced with a heteroatom such as —O—, —N—, —S—, —S(O)ₙ- (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or =S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —(S)OR⁷⁰, —C(O)NR⁸⁰R⁸⁰, —C(NR⁷⁰)NR⁸⁰R⁸⁰, —OC(O)R⁷⁰, —C(S)R⁷⁰, —C(O)O⁻M⁺, —C(O)OR⁷⁰, —C(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰R⁷⁰ and —NR70C(NR70)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R^{80′}s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁—C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

The terms “detectable moiety” and “detectable tag” are used interchangeably herein to refer to a tag, moiety, and/or molecule which has properties that can be detected and/or measured, directly or indirectly. Detectable tags of interest include, but are not limited to, an enzyme, a nucleic acid, a polypeptide, a particle, an affinity tag (e.g., an antigen, a hapten or a member of a specific binding pair such as a biotin moiety), a fluorophore, a chromophore, a luminescent tag, a radioactive tag or a chemoselective tag.

As used herein, a “member of a specific binding pair” is a member of a pair of molecules or entities that takes part in a specific binding interaction. Where a first member of the specific binding pair is identified, the identity of the second member of the specific binding pair may be readily identifiable. It should be noted that when either member of the binding pair is referred to as the first member, the remaining member is understood to be the second member and vice versa. Examples of specific binding pair interactions include immune interactions such as antigen/antibody and hapten/antibody as well as non-immune interactions such as complementary nucleic acid binding, complementary protein-protein interactions, a sugar and a lectin specific therefore, an enzyme and an inhibitor therefore, an apoenzyme and cofactor, a hormone and a receptor therefore, biotin/avidin and biotin/streptavidin.

As used herein, the term “biotin moiety” refers to an affinity agent that includes biotin or a biotin analogue such as desthiobiotin, oxybiotin, 2′-iminobiotin, diaminobiotin, biotin sulfoxide, biocytin, etc. Biotin moieties bind to streptavidin with an affinity of at least 10⁻⁸ M. A biotin affinity agent may also include a linker, e.g., -LC-biotin, -LC-LC-Biotin, -SLC-Biotin or -PEGₙ-Biotin where n is 3-12.

The terms “fluorophore” and “fluorescent probe” refer to a molecule that, when excited with light having a selected wavelength, emits light of a different wavelength, which may emit light immediately or with a delay after excitation. Fluorophores, include, without limitation, fluorescein dyes, e.g., 5-carboxyfluorescein (5-FAM), 6-carboxyfluorescein (6-FAM), 2′,4′,1,4,-tetrachlorofluorescein (TET), 2′,4′, 5′,7′,1,4-hexachlorofluorescein (HEX), and 2′,7′-dimethoxy-4′,5′-dichloro-6-carboxyfluorescein (JOE); cyanine dyes, e.g. Cy3, CY5, Cy5.5, QUASARTM dyes etc.; dansyl derivatives; rhodamine dyes e. g. 6-carboxytetramethylrhodamine (TAMRA), CAL FLUOR dyes, tetrapropano-6-carboxyrhodamine (ROX). BODIPY fluorophores, ALEXA dyes, Oregon Green, pyrene, perylene, benzopyrene, squarine dyes, coumarin dyes, luminescent transition metal and lanthanide complexes and the like. The term fluorophore includes excimers and exciplexes of such dyes.

The terms “amino acid analog,” “unnatural amino acid,” and the like may be used interchangeably, and include amino acid-like compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y) Amino acid analogs also include natural amino acids with modified side chains or backbones Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or combinations thereof. For example, amino acid analogs may include α-hydroxy acids, and α-amino acids, and the like.

The term “carbohydrate” and the like may be used to refer to monomers units and/or polymers of monosaccharides, disaccharides, oligosaccharides, and polysaccharides. The term sugar may be used to refer to the smaller carbohydrates, such as monosaccharides, disaccharides. The term “carbohydrate derivative” includes compounds where one or more functional groups of a carbohydrate of interest are substituted (replaced by any convenient substituent), modified (converted to another group using any convenient chemistry) or absent (e.g., eliminated or replaced by H). A variety of carbohydrates and carbohydrate derivatives are available and may be adapted for use in the subject compounds and conjugates.

The term “glycan” is used to refer to the carbohydrate portion of a glycoconjugate, such as a glycoprotein, glycolipid, a glycopolymer, or a proteoglycan. In some cases, glycans consist solely of O-glycosidic linkages of monosaccharides. For example, cellulose is a glycan (or, to be more specific, a glucan) composed of β-1,4-linked D-glucose, and chitin is a glycan composed of β-1,4-linked N-acetyl-D-glucosamine Glycans can be homo- or heteropolymers of monosaccharide residues, and can be linear or branched.

The term “glycocalyx” refers to a glycoprotein-polysaccharide layer or coating that surrounds the cell membranes of some bacteria, epithelia and other cells. This coating consists of several carbohydrate moieties of membrane glycolipids and glycoproteins, which serve as backbone molecules for support. In some instances, the carbohydrate portion of the glycolipids found on the surface of plasma membranes helps these molecules contribute to cell-cell recognition, communication, and intercellular adhesion. The glycocalyx is a type of identifier that the body uses to distinguish between its own healthy cells and transplanted tissues, diseased cells, or invading organisms. Included in the glycocalyx are cell-adhesion molecules that enable cells to adhere to each other and guide the movement of cells during embryonic development.

The term “mucin” refers to a family of high molecular weight, heavily glycosylated proteins produced by epithelial tissues in most organisms. A mucin can form a gel and is a key component in most gel-like secretions, serving functions from lubrication to cell signaling to forming chemical barriers.

The term “antibody” is used in the broadest sense and includes monoclonal antibodies (including full length monoclonal antibodies), polyclonal antibodies, and multispecific antibodies (e.g., bispecific antibodies), humanized antibodies, single-chain antibodies, chimeric antibodies, antibody fragments (e.g., Fab fragments), and the like. An antibody is capable of binding a target antigen. (Janeway, C., Travers, P., Walport, M., Shlomchik (2001) Immuno Biology, 5th Ed., Garland Publishing, New York). A target antigen can have one or more binding sites, also called epitopes, recognized by complementarity determining regions (CDRs) formed by one or more variable regions of an antibody.

The term “natural antibody” refers to an antibody in which the heavy and light chains of the antibody have been made and paired by the immune system of a multi-cellular organism. Spleen, lymph nodes, bone marrow and serum are examples of tissues that produce natural antibodies. For example, the antibodies produced by the antibody producing cells isolated from a first animal immunized with an antigen are natural antibodies.

The term “humanized antibody” or “humanized immunoglobulin” refers to a non-human (e.g., mouse or rabbit) antibody containing one or more amino acids (in a framework region, a constant region or a CDR, for example) that have been substituted with a correspondingly positioned amino acid from a human antibody. In general, humanized antibodies produce a reduced immune response in a human host, as compared to a non-humanized version of the same antibody. Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332). In certain embodiments, framework substitutions are identified by modeling of the interactions of the CDR and framework residues to identify framework residues important for antigen binding and sequence comparison to identify unusual framework residues at particular positions (see, e.g., U.S. Pat. No. 5,585,089; Riechmann et al., Nature 332:323 (1988)). Additional methods for humanizing antibodies contemplated for use in the present invention are described in U.S. Pat. Nos. 5,750,078; 5,502,167; 5,705,154; 5,770,403; 5,698,417; 5,693,493; 5,558,864; 4,935,496; and 4,816,567, and PCT publications WO 98/45331 and WO 98/45332. In particular embodiments, a subject rabbit antibody may be humanized according to the methods set forth in U520040086979 and U520050033031. Accordingly, the antibodies described above may be humanized using methods that are well known in the art.

The term “chimeric antibodies” refer to antibodies whose light and heavy chain genes have been constructed, typically by genetic engineering, from antibody variable and constant region genes belonging to different species. For example, the variable segments of the genes from a mouse monoclonal antibody may be joined to human constant segments, such as gamma 1 and gamma 3. An example of a therapeutic chimeric antibody is a hybrid protein composed of the variable or antigen-binding domain from a mouse antibody and the constant or effector domain from a human antibody, although domains from other mammalian species may be used.

“Treatment” refers to both therapeutic treatment and prophylactic or preventative measures. Those in need of treatment include those already with the disorder as well as those in which the disorder is to be prevented. As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e g , in a human or in a non-human mammal, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines), etc. “Subject” for purposes of treatment refers to any animal classified as a mammal, including humans, domestic and farm animals, and zoo, sports, or pet animals, such as dogs, horses, cats, cows, etc. In some cases, the subject is human.

Other terms used herein and in the claims adopt their plain meanings as would have been understood by one of skill in the relevant art, that are not inconsistent with the usages in the instant specification. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques of described herein are those well known and commonly used in the art.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a lipid conjugate” includes a plurality of such lipid conjugates and reference to “the lipid conjugate” includes reference to one or more lipid conjugates and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

As summarized above, the present disclosure includes methods of labelling a cell. The subject methods can include contacting the cell with a cholesteryl-cargo conjugate to non-covalently bind the cholesterylamine anchoring group to the cell membrane thereby displaying a linked cargo moiety at the cell surface for an extended period of time.

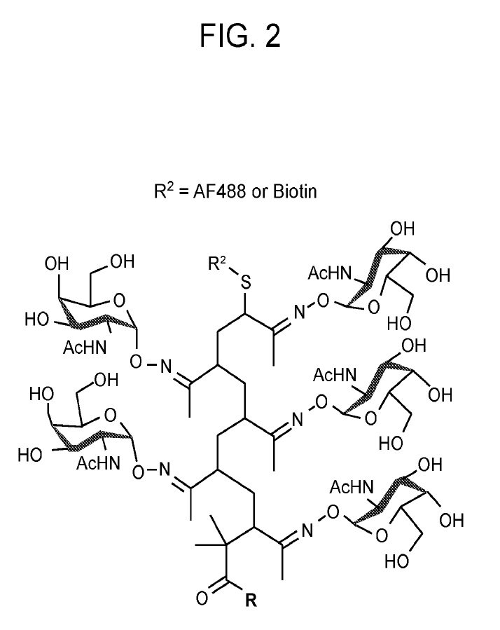

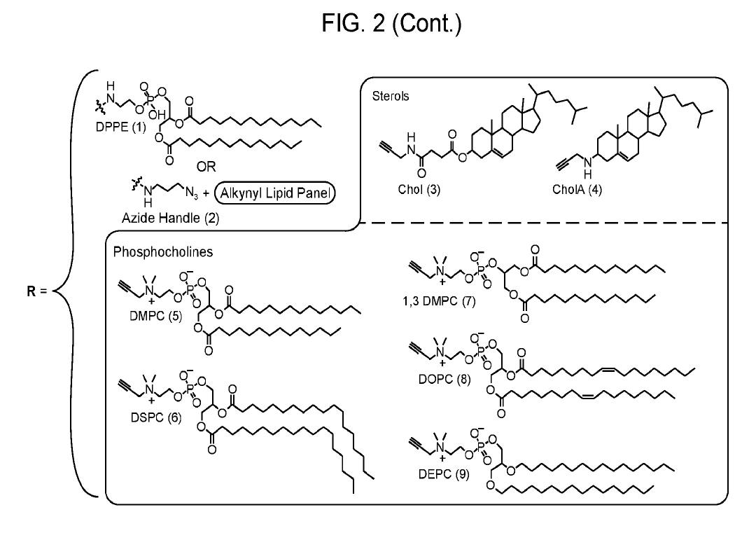

FIG. 2, panel A, shows a lipid conjugate functionalized with a dipalmitoyl phosphatidylethanolamine-based anchor (DPPE). Such lipid conjugates can be constitutively internalized by cells. As a consequence, their plasma membrane residence half-lives are short (e.g., up to 5 or 6 hours), which limits the application of such lipid conjugates to studies of biological processes occurring on short time scales. However, biological events such as tumor growth and metastasis, unfold on much longer timescales of days to weeks. For example, the role of glycosylation in such processes cannot be addressed using short-lived conjugates via lipid-insertion-based glycocalyx engineering.

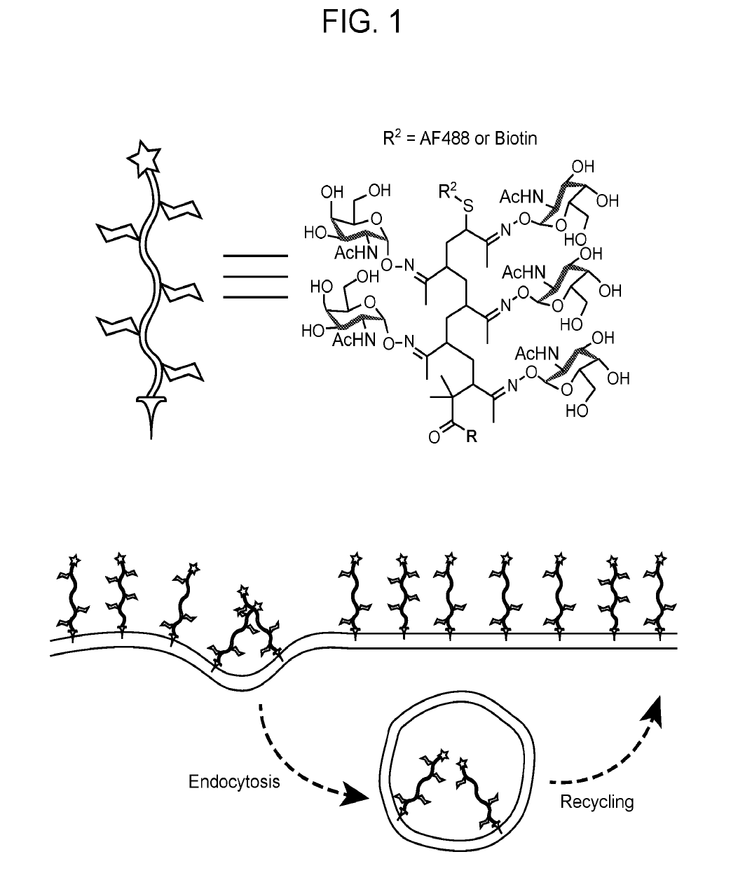

FIG. 1 shows a schematic of a mechanism by which the subject cholesterylamine-anchored cargo moieties (e.g., glycopolymer cargo moieties) are endocytosed back to the cell surface and displayed at the surface of the cell membrane for an extended period of time. The synthetic lipid cholesterylamine is conjugated to the cargo moiety to be displayed on the cell surface. The target cell and the conjugate are combined in solution. The lipid conjugate enters the plasma membrane driven by hydrophobic interactions, and the cargo moiety is thus displayed on the cell surface. Because all cells have plasma membranes, any cell type can be labelled with a desired cargo moiety (e.g., any cells can be used: primary cells, ex vivo cells, precious/rare cells etc.) and no additional engineering of the cells is needed.

The present disclosure provides for lipid constructs that are internalized but recycled to the cell surface to provide for the continuous display of the linked cargo moieties (e.g., glycopolymers) at the cell surface for an extended period of time. In this way, any cargo molecule of interest could be added to any target cell and the cargo will persist for an extended period of time, e.g., from days to weeks, e.g., up to 10 days. This persistent display of cargo moieties on the surface-engineered cell finds use in a variety of applications where it is desirable to have a particular cargo moiety presented on the cell surface. Any convenient entities (e.g., biochemical entities, small molecules, etc.) can be engineered into the subject lipid conjugates and displayed on a target cell to modulate any biological process that relies on the cell surface as a site of action.

Other noncovalent cell-surface engineering technologies are limited by endocytosis (internalization) of the molecules introduced onto the cell. The present disclosure provides conjugates with a lipid anchor that, after internalization, is capable of recycling its cargo back to the cell surface resulting in a steady-state presentation of the linked cargo moiety for extended periods of time, even through rounds of cell division.

Applications of interest include, but are not limited to, protection from the immune system, recruitment of the immune system, exploitation of various cellular adhesion properties, and exploitation of various cell-cell interactions.

Cholesterylamine Conjugates

Aspects of the present disclosure include cholesterylamine conjugates. In some embodiments, the cholesterylamine conjugate has the formula:

C-L-Z (I)
wherein:
C is a cholesterylamine anchor group;
L is an optional linker (e.g., a cleavable or a non-cleavable linker); and
Z is a linked cargo moiety for delivery to a cell surface.

A cholesterylamine anchor group is a derivative of cholesterylamine or an analog thereof. In one aspect, the cholesterol derivative is a cholesteryl, a dihydrocholesteryl, or a sitosterol groups, where the group includes an amino substituent, usually at the 3-position of the steroid. In some embodiments, the conjugate includes a cholesterylamine anchor group that is an amine-linked cholesterylamine, e.g., a cholesterylamine substituted at the 3-amino group to form an amino linkage to a linked cargo moiety of interest. In certain embodiments, the conjugate includes a dihydrocholesteryl-containing anchor group. In certain embodiments, the conjugate includes a sitosterol-containing anchor group. In some embodiments, the cholesterylamine conjugate is described by the formula (II):

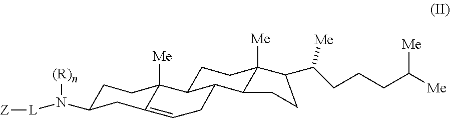

wherein L and Z are as described for formula (I), each R is independently selected from H, an alkyl (e.g., methyl or ethyl) and a substituted alkyl and n is 1 or 2, or a salt thereof. In certain embodiments of formula (II), L is selected such that the 3-amino group of the cholesterylamine is a secondary (—NH—), tertiary (—NR—) or quaternary (—NR₂⁺—) amino group, such that the linking 3-amino group can be positively charged, e.g., under aqueous conditions, and form a salt, e.g., as salt —NH₂⁺—X⁻. In some instances of formula (II), n is 2 and each R is H, such that the N is positively charged. It is understood that any of the cholesterylamine conjugate structures described herein may optionally be present in a salt form.

In some embodiments, the cholesterylamine conjugate is described by the formula (III):

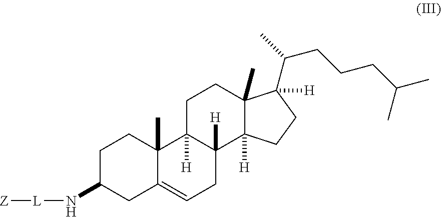

wherein L and Z are as described for formula (I). It is understood that the cholesterylamine structure can be represented in a variety of ways, see e.g., the stereochemical representations of formulae (II) and (III).

A cholesterylamine conjugate can be prepared according to the methods described by Peterson is U.S. Pat. No. 8,637,468, the disclosure of which is herein incorporated by reference in its entirety.

Any convenient linking groups may be utilized in the subject conjugates. In certain embodiments, L includes a polymer. For example, the polymer may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In certain embodiments, the polymer is a polyalkylene glycol. In certain embodiments, the polymer is a polyethylene glycol.

In some embodiments, L is a linker comprising —(L¹)ₐ—(L²)_{b}—(L³)_{c}—(L⁴)_{d}—(L⁵)ₑ—, wherein L¹, L² , L³, L⁴ and L⁵ are each a linker unit, and a, b, c, d and e are each independently 0 or 1, wherein the sum of a, b, c, d and e is 1 to 5. Linker units of interest include those subunits shown in the conjugates and compounds described in more detail herein. Linker units of interest include, but are not limited to (C₁-C₁₂)alkyl, substituted (C₁-C₁₂)alkyl, ethylene diamine, PEG, amino acids, polypeptides, para-amino-benzyloxycarbonyl (PABC), a meta-amino-benzyloxycarbonyl (MABC), a para-amino-benzyloxy (PABO), a meta-amino-benzyloxy (MABO), para-aminobenzyl, an acetal group, a disulfide, a hydrazine, a protease-cleavable moiety, a glucuronidase cleavable moiety, a beta-lactamase cleavable moiety and an ester.

In certain embodiments, the linker L is described by one of the following structures:

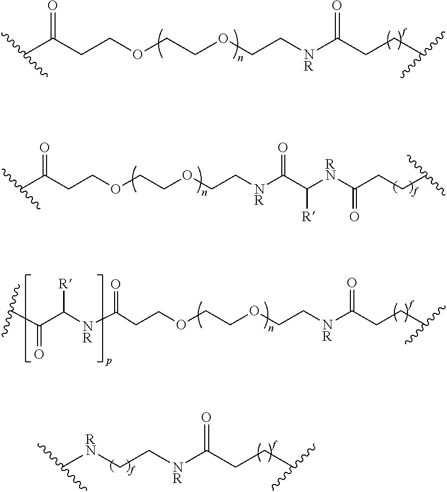

wherein: each f is independently 0 or an integer from 1 to 12;

each n is independently 0 or an integer from 1 to 30;

each p is independently 0 or an integer from 1 to 20;

each R is independently hydrogen, alkyl or substituted alkyl; and

each R′ is independently H, a sidechain group of an amino acid, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain instances, each R′ is a sidechain group of a naturally occurring amino acid.

In some embodiments, the cholesterylamine conjugate is described by the formula (IV):

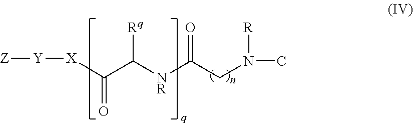

wherein Z and C are as described for formula (I);

X is absent or a coupling group;

Y is absent or a linking moiety;

n is 1, 2, 3, 4 or 5; and

q is 0 or an integer from 1 to 20, wherein each Rq is independently an amino acid sidechain group.

In some embodiments, the cholesterylamine conjugate is described by the formula (V):

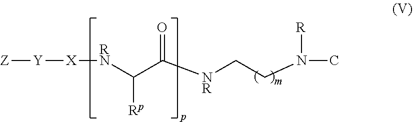

wherein Z and C are as described for formula (I);

X is absent or a coupling group;

Y is absent or a linking group;

n is 1, 2, 3, 4 or 5;

each R is independently H, an alkyl (e.g., methyl or ethyl) or a substituted alkyl; and

q is 0 or an integer from 1 to 20, wherein each Rq is independently an amino acid sidechain group.

The amino acid residues of formulae (IV) and (V) can be one or more natural or non-natural amino acids, essential amino acids, or non-essential amino acids, or derivatives of amino acids having L or D configuration. In some embodiments of formulae (IV) and (V), the linker Y is selected from a straight chain or branched or cyclic substituted or unsubstituted alkyl group having C₁-C₁₀₀, a polypeptide, a polynucleotide, polysaccharide, a polyethylene glycol, a biodegradable linker, or combinations thereof.

In some embodiments of formulae (IV) and (V), the coupling group X includes an amide, ether, ester, carbamate, alkyl, aryl, alkene, triazole, amine, or alkanol. In certain instances, the coupling group X is derived from a coupling reaction between the linker and a coupling agent selected from a dithio diacid, a dicarboxylic acid, an acrylic moiety, a diazide, a styrene, a vinyl carboxylic acid, a urethane, a vinyl acetate, a vinyl ether, a Diels-Alder reagent, disulfides, hydrazones, imines, acetals, orthoesters, or other acid-labile or redox sensitive groups that allow release of agents in cells or tissues, photopolymerizable moiety, derivatives thereof, and combinations thereof. Dithio diacids of interest include, but are not limited to, dithio dicarboxylic acid, dithio dipropanoic acid, dithio dibutanoic acid, dithio dipentanoic acid, dithio dihexanoic acid, and derivatives and combinations thereof. Specific examples of dithio diacids can include 16-carboxyhexadecyl disulfide, 5,5′ dithiobis(2-nitrobenzoic acid), 2,2′-dithiodibenzoic acid, 4,4′-dithiodibutyric acid, 3,3′-dithiodipropionic acid and 6,6′-dithiodinicotinic acid. Dicarboxylic acids of interest include, but are not limited to, oxalic acid, malonic acid, succinic acid, glutaric acid, adipic acid, pimelic acid, suberic acid, azelaic acid, sebacic acid, phthalic acid, maleic acid, isophthalic acid, terephthalic acid, and derivatives and combinations thereof.

In some cases, the linker is reversibly cleavable. For example, linking with a dithio containing group can be reversed with the addition of dithiothreitol (DTT) or a similar reducing reagent that can break the disulfide linkage in the linker. The cross-linking can be reinitiated by addition of an oxidizing agent such as, but not limited to, hydrogen peroxide.

In certain instances, the Y linker and/or X coupling group can be prepared by “click” reactions, such as a 1,3 dipolar cycloaddition of an azide and alkyne or similar condensation to yield a triazole or other linking subunit. In some embodiments, the coupling group X is derived from a reaction with hydroxybenzotriazole (HOBt) and a carbodiimide reagent. Suitable examples of carbodiimide reagents include, but are not limited to, N,N′-dicyclohexylcarbodiimide (DCC), N,N′-diisopropylcarbodiimide (DIC), 1-Ethyl-3-(3-dimethylaminopropyl)carbodiimide (EDC), and combinations thereof.

In some instances, the cholesterylamine conjugate is prepared via the coupling of a cholesterylamine-linked conjugation tag with a cargo moiety having a compatible reactive functional group. As used herein the term “conjugation tag” refers to a group that includes a chemoselective functional group (e.g., as described herein) that can covalently link with a compatible functional group of a target molecule, after optional activation and/or deprotection. Any convenient conjugation tags may be utilized in preparation of the subject lipid conjugates in order to conjugate the lipid to a target cargo moiety of interest. In some embodiments, the conjugation tag includes a terminal functional group selected from an amino, a carboxylic acid or a derivative thereof, a thiol, a hydroxyl, a hydrazine, a hydrazide, an azide, an alkyne and a protein reactive group (e g amino-reactive, thiol-reactive, hydroxyl-reactive, imidazolyl-reactive or guanidinyl-reactive).

Any convenient methods and reagents can be adapted in order to covalently link the conjugation tag to the target cargo moiety. Methods of interest for preparation of the lipid conjugates, include but are not limited to, those methods and reagents described by Hermanson, Bioconjugate Techniques, Third edition, Academic Press, 2013. The conjugating may be performed in an aqueous solution. In some instances, the conjugation tag includes an amino functional group and the target cargo moiety includes an activated ester functional group, such as a NHS ester or sulfo-NHS ester, or vice versa. In certain instances, the conjugation tag includes a maleimide functional group and the target cargo moiety includes a thiol functional group, or vice versa. In certain instances, the conjugation tag includes an alkynyl functional group and the target cargo moiety includes an azide functional group, or vice versa.

### Cargo Moieties

Any convenient cargo moieties of interest for display at a cell surface may be included in the subject lipid conjugates. Types of cargo moieties of interest for delivery to a cell surface include, but are not limited to, nucleic acids, carbohydrates, polymers (e.g., a glycopolymer), peptides, small molecules, drugs, prodrugs, proteins, epitopes and combinations thereof. In some embodiments, the cargo moiety is a nucleic acid, e.g., an aptamer.

In some embodiments, the cargo moiety is selected from an immuno-inhibiting agent, an immuno-activating agent, an immuno-modulating agent, an adhesion modulating agent (e.g., adhesion promoting or inhibiting agent), a cell surface displayed therapeutic agent, a targeting agent, a cell differentiation agent and an imaging agent.

In some embodiments, the cargo moiety is an immuno-inhibiting agent. As used herein, by the terms “immune inhibiting” and “immuno-inhibiting” are used interchangeably and refer to an agent that inhibits at least one response (an innate response or an adaptive response) of the subject's immune system. Examples of an immune system response that may be inhibited by an immune inhibiting agent include, but are not limited to, NK cell activity, phagocytosis, cytokine production, antibody production, cytotoxicity response, and production of humoral factors. Examples of immune inhibiting agents include steroids, retinoic acid, dexamethasone, cyclophosphamide, anti-CD3 antibody or antibody fragment, and other immunosuppressants. Any convenient immuno-inhibiting agents may be utilized in the cargo moieties of the subject lipid conjugates. In certain embodiments, the immuno-inhibiting agent is selected from an immuno-inhibiting glycan, an immuno-inhibiting glycopolymer, an immuno-inhibiting protein, an immuno-inhibiting small molecule and an immuno-inhibiting peptide. In certain instances, the immuno-inhibiting agent comprises a sialic acid containing glycan, such as Sia, N-acetylneuraminic acid; lactose, galactose-b1,4-glucose; GalNAc, N-acetylgalactosamine; GD3, N-acetylneuraminic acid-α2,8-N-acetylneuraminic acid-α2,3-galactose-β1,4-glucose; or SiaLex, N-acetylneuraminic acid-α2,3-galactose-β1,4-(α1,3-fucose)-N-acetylglucosamine

In some embodiments, the cargo moiety is an immuno-activating agent. As used herein, by the terms “immune activating” and “immuno-activating” are used interchangeably and refer to an agent that activates at least one response (an innate response or an adaptive response) of the subject's immune system. Examples of an immune system response that may be activated by an immune activating agent include, but are not limited to, NK cell activation, activation of phagocytosis, cytokine activation, antibody production, cytotoxicity response, and activation of humoral factors such as complement activation. In some cases, an immune-activating agent is an antibody (e.g., a humanized antibody; a humanized monoclonal antibody; a single-chain Fv; etc.); for example, anti-CTLA4, anti-PD-1, anti-PD-L1, anti-PD-L2, anti-CD137, anti-TGF-β1, anti-TGF-β2, anti-TGF-β3, etc., antibody. In some cases, an immune-activating agent is a cytokine or a chemokine. Exemplary cytokines and chemokines include, but are not limited to, IL-2, IL-7, IL-12, IL-15, IL-21, GM-CSF, IFN-α, and CCL-21 Any convenient immuno-activating agents may be utilized in the cargo moieties of the subject lipid conjugates. In certain embodiments, the immuno-activating agent is selected from an immuno-activating glycans, an immuno-activating glycopolymers, an immuno-activating protein, an immuno-activating small molecule and an immuno-activating peptide. In certain instances, the immuno-activating agent is a fungal glycan, e.g., a poly- or bi-mannoside, which activates an immune response. In certain instances, the subject conjugate includes an immuno-activating agent capable of recruiting and activating the immune system in situ.

In certain embodiments, the cargo moiety is an immuno-modulating agent. As used herein, the terms “immuno-modulating” and “immune modulating” are used interchangeably and refer to an agent that changes the polarization (i.e. type of activity) of an immune system cell (e.g., a macrophage or other cell) in target tissues. This immune modulating activity is distinct from immune inhibition or activation. The immune cells are still active, but their roles can change from ‘kill foreign/transformed cells’ to ‘heal this tissue’. Any convenient immuno-modulating agents may be utilized in the cargo moieties of the subject lipid conjugates. In certain embodiments, the immuno-modulating agent is selected from an immuno-modulating glycan, an immuno-modulating glycopolymer, an immuno-modulating peptide, an immuno-modulating small molecule and an immuno-modulating protein. In certain cases, the immuno-modulating agent is a sialic acid-containing glycan capable of changing the polarization of macrophages in a target tissue. In certain instances, the immuno-modulating agent can modulate inflammation. In some cases, the immuno-modulating agent is an anti-inflammatory agent.

In certain embodiments, the cargo moiety includes an adhesion modulating agent. As used herein, the term “adhesion modulating” refers to an agent that promotes or inhibits adhesion of a cell to any convenient substance, such as to a tissue or an extracellular matrix in vivo. Any convenient adhesion modulating agents may be utilized in the cargo moieties of the subject lipid conjugates. In certain embodiments, the adhesion modulating agent is selected from an adhesion modulating glycan, an adhesion modulating glycopolymer, an adhesion modulating protein, an adhesion modulating small molecule and an adhesion modulating peptides. In some cases, an adhesion modulating agent finds use in the subject labelled cells in a cell-based therapy to adhere better the cell to its target tissue. For example, stem cells are, in general, susceptible to anchorage-dependent cell death, such that if these cells don't bind extracellular matrix, the cells can die. The subject glycopolymers can improve the adhesion of target cells to the extracellular matrix.

In some embodiments, a therapeutic labelled cell needs to be maintained in the blood for the cell to be effective. In such cases, it may be desirable to utilize an adhesion modulating agent which inhibits adhesion of the labelled cell. For example, display of oligomeric RGD peptides on the labelled cell can block the cells from binding their natural substrate in the extra cellular matrix, where the integrins (the proteins responsible for adhesion) would bind those RDG peptides in cis and be blocked. In certain embodiments, the cargo moiety includes an adhesion inhibiting agent. In certain embodiments, the cargo moiety includes an adhesion promoting agent.

In certain embodiments, the cargo moiety is a cell surface displayed therapeutic agent. Any convenient therapeutic agent which is desirable to display on the outside of a target cell can be utilized as a cargo moiety. In certain instances, the therapeutic agent is an agent that is undesirable to be endocytosed inside the cell. In some instances, the therapeutic agent is a therapeutic protein. In some instances, the therapeutic agent is a peptide. In some cases, the therapeutic agent is a small molecule. One could have a targeting cargo and a therapeutic cargo on a cell, and the cell acts as the delivery mechanism to the target tissue. In certain embodiments, the labelled cell displays a therapeutic agent that is linked to the cholesterylamine anchoring group via a cleavable linker. As such, the therapeutic agent can be cleaved from the cell, upon application of a convenient stimulus or environmental condition.

In certain embodiments, the cargo moiety includes a targeting agent. As used herein, a targeting agent is an agent that acts to direct a cell on which it is displayed to a particular site in vivo, e.g., by specifically binding with a member of a specific binding pair located at the site, or chemoselectively reacts with a compatible reactive functional group located at the site. Any convenient targeting agent can be utilized as a cargo moiety. In some embodiments, the targeting agent is a glycan. In some embodiments, the targeting agent is a glycopolymers. In some embodiments, the targeting agent is a protein. In some embodiments, the targeting agent is a peptide. In some embodiments, the targeting agent is a small molecule. In some embodiments, the targeting agent is selected from antibodies, antibody fragments, nucleic acids (such as aptamers, and others), and exogenously expressed receptors. In certain instances, the cargo moiety includes a targeting agent genetically fusion to the Fc portion of an antibody of interest. For example, a targeting protein or glycan can play a role in the directing/homing of, for example immune cells, to inflamed tissues. The endothelium in the blood vessels surrounding inflamed tissue express ligands and/or receptors so that the immune cells circulating know that that is the site at which to leave the circulation and enter the tissue.

A variety of different cells may be directed to a variety of different tissues by display of a targeting agent. In some instances, the labelled cell including a targeting agent, has itself a desirable biological activity at the delivery site in vivo. In some instances, the labelled cell including a targeting agent, further includes a second cholesterylamine conjugate having a cargo moiety that provides a desirable biological activity at the delivery site in vivo. In some instances, the labelled cell including a targeting agent, further includes a therapeutic agent. For example, any convenient immune system cells can be targeted to an inflamed or an infected tissue via display of a suitable targeting agent. In some instances, the labelled cell is an immune cell and the targeting agent directs the cell to an inflamed tissue. In some instances, the labelled cell is an immune cell and the targeting agent directs the cell to an inflamed tissue.

Immune cells of interest which may be labelled according to the subject methods include, but are not limited to, B cells, T cells, NK cells, macrophages, neutrophils, monocytes, platelets, and red blood cells (RBCs).

In some embodiments, the labelled cell is a beta-islet cell and the targeting agent is a pancreas-targeting agent that directs the labelled cell to the pancreas. In some embodiments, the labelled cell is a cell engineered to produce glycosidases. In certain instances, the cell engineered to produce glycosidases is labelled to include a targeting agent that directs the cell to the blood brain barrier (BBB). Administration of glycosidase-producing cells labelled with a targeting agent to direct the cells to the BBB, finds use in the treatment of subjects with lysosomal storage diseases (i.e. subjects who genetically lack those glycosidases, which leads to accumulation of gangliosides in neurons and brain damage).

In certain embodiments, the cargo moiety includes a cell differentiation agent. Any convenient cell differentiation agents can be utilized in the cargo moiety. In some embodiments, the cell differentiation agent is a cell differentiating glycan. In some embodiments, the cell differentiation agent is a cell differentiating glycopolymers. In some embodiments, the cell differentiation agent is a cell differentiating peptide. In some embodiments, the cell differentiation agent is a cell differentiating protein. In some embodiments, the cell differentiation agent is a cell differentiating small molecule. In some cases, a cell-surface glycan induces a target stem cell to undergo directed differentiation into specific lineages (e.g. neuronal cells).

In some embodiments, the labelled cells are stem cells which are first grown in a lab, optionally in combination with a targeting agent cargo moiety, before labelling with a cell differentiating glycan on their cell surfaces before introducing them into the patient. When the labelled cells reach the target tissue, they can start differentiating into the desired tissue/cell type. In some cases, this method of delivery would be more feasible, for example, than trying to deliver neurons themselves, as you would want the developing neurons to establish connections with surrounding neurons, and a differentiated neuron is less plastic than a differentiating neuron.

In certain embodiments, the cargo moiety is an imaging agent. Any convenient imaging agents can be utilized as a cargo moiety in the subject cholesterylamine conjugates. In some embodiments, the cargo moiety is a fluorescent probes (e.g., as described herein). In some embodiments, the cargo moiety is an IR fluorescent dye. In some embodiments, the cargo moiety is a PET emitting probe, such as fluorodeoxyglucose F18, [18F]maltose, [18F]maltohexaose, [18F]2-fluorodeoxysorbitol (FDS) or other probe including a radioactive tracer). In some embodiments, the cargo moiety is an NMR contrast agent, such as a gadolinium(III) chelate containing contrast agent, e.g., dotarem, omniscan, and the like.

In some embodiments, the cargo moiety includes a glycan structure of interest. In some instances, the cargo moiety is a glycopolymer that includes multiple linked glycans. Any convenient glycans that find use in a cell glycocalyx or an engineered cell glycocalyx may be incorporated into the subject lipid conjugates. Glycans of interest include, but are not limited to those described by Hudak et al., Nature Chemical Biology, 10, Jan. 2014, 69-75, such as Sia, N-acetylneuraminic acid; lactose, galactose-b1,4-glucose; GalNAc, N-acetylgalactosamine; GD3, N-acetylneuraminic acid-α2,8-N-acetylneuraminic acid-α2,3-galactose-β1,4-glucose; and SiaLex, N-acetylneuraminic acid-α2,3-galactose-β1,4-(α1,3-fucose)-N-acetylglucosamine

In some embodiments, the cargo moiety includes a glycan described by the structure:

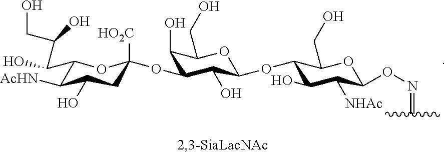

In some embodiments, the cargo moiety includes a glycan described by the structure:

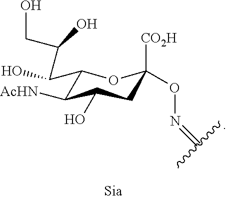

In some embodiments, the cargo moiety includes a glycan described by the structure:

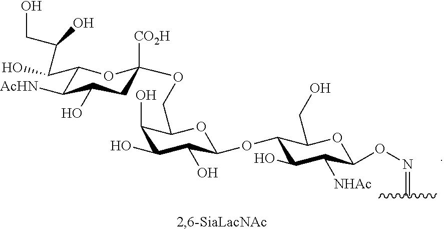

In some embodiments, the cargo moiety includes a glycan described by the structure:

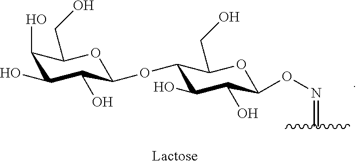

In some embodiments, the cargo moiety includes a glycan described by the structure:

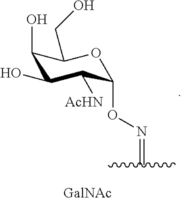

In some embodiments, the cargo moiety includes a glycan described by the structure:

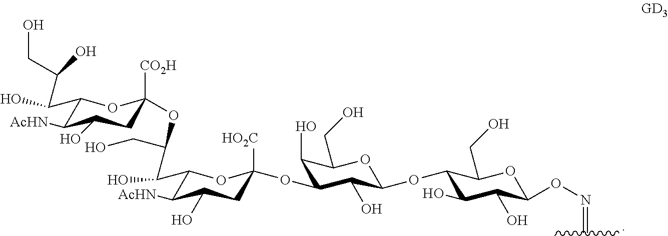

In some embodiments, the cargo moiety includes a glycan described by the structure:

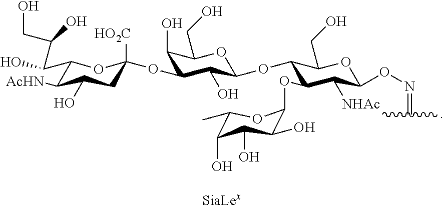

In some embodiments, the cargo moiety includes a acetic acid group described by the structure:

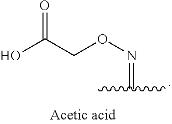 [SMILES: *=NOCC(=O)O]

In some embodiments, the cargo moiety includes a glycerol group described by the structure:

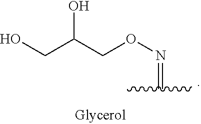 [SMILES: *=NOCC(O)CO]

In some embodiments, one or more of the glycans and groups described above are attached to a polymer to provide a glycopolymer cargo moiety. Any convenient glycans and groups may be attached to the sidechains of a convenient polymeric backbone to provide an engineered glycopolymer. When such glycopolymers are used to label a cell according to the subject methods, an engineered glycocalyx may be formed around the target cell. Selection of particular glycans and other sidechain groups provides for a desirable engineered glycocalyx which imparts on the target cell a desired biological activity or property. In some instances, the engineered glycocalyx may form a mucin-like layer around the target cell.

In certain embodiments, a glycopolymer cargo moiety comprises a methyl vinyl ketone polymer where the ketone groups are coupled with a glycan (e.g., as described herein), such as an aminooxyglycan, or an aminooxy compound (see e.g., the glycopolymer structures described by Hudak et al., Nature Chemical Biology, 10, Jan. 2014, 69-75). In certain embodiments, a glycopolymer cargo moiety is described by the following formula (VI):

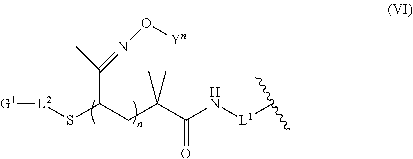

wherein each Y is independently a glycan or sidechain group (e.g., as described herein);
L¹ and L² are each optional linkers;
n is an integer from 1 to 10,000; and
G¹ is selected from H, an alkyl, a substituted alkyl and a detectable moiety. It is understood that when the group Y is a glycan, the adjacent O atom can be derived from the glycan itself.

In certain embodiments of formula (VI), each Y is independently selected from a monosaccharide, a disaccharide, a trisaccharide, a tetrasaccharide, acetic acid and glycerol. In certain embodiments of formula (VI), each Y is independently selected from Sia, N-acetylneuraminic acid; lactose, galactose-b1,4-glucose; GalNAc, N-acetylgalactosamine; GD3, N-acetylneuraminic acid-α2,8-N-acetylneuraminic acid-α2,3-galactose-β1,4-glucose; SiaLex, N-acetylneuraminic acid-α2,3-galactose-β1,4-(α1,3-fucose)-N-acetylglucosamine, acetic acid and glycerol. In certain embodiments of formula (VI), at least one Y is selected from Sia, lactose, GalNAc, GD3; SiaLex, acetic acid and glycerol. In certain embodiments of formula (VI), each Y is Sia, N-acetylneuraminic acid. In certain embodiments of formula (VI), each Y is lactose, galactose-β1,4-glucose. In certain embodiments of formula (VI), each Y is GalNAc, N-acetylgalactosamine. In certain embodiments of formula (VI), each Y is GD3, N-acetylneuraminic acid-α2,8-N-acetylneuraminic acid-α2,3-galactose-β1,4-glucose. In certain embodiments of formula (VI), each Y is SiaLex, N-acetylneuraminic acid-α2,3-galactose-β1,4-(α1,3-fucose)-N-acetylglucosamine In certain embodiments of formula (VI), at least one Y is acetic acid or glycerol.

In certain embodiments of formula (VI), at least one Y is described by the following structure:

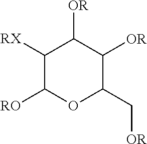

wherein X is O or NHAc and each R is H, an alkyl, a substituted alkyl, a monosaccharide, a disaccharide and a trisaccharide, wherein at least one R group is linked to the polymer backbone.

In certain embodiments of formula (VI), the glycopolymer cargo moiety Z has the following structure:

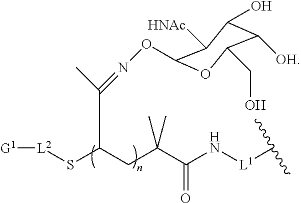

### Cells

Any convenient cells may be labelled according to the subject methods. Suitable cells include prokaryotic cells and eukaryotic cells. In some cases, the cell is a eukaryotic cell. In some cases, the cell is in vitro. In some cases, the cell is ex vivo. In some cases, the cell is in vivo. In some cases, the cell is an in vitro eukaryotic cell. In some cases, the cell is an ex vivo eukaryotic cell. In some cases, the cell is an in vivo eukaryotic cell, e.g., an in vivo mammalian cells. In some cases, the cell is a prokaryotic cell, e.g., a bacterial cell.

In some cases, the cell is a eukaryotic cell. In some embodiments, the cell is selected from an immune cell, a stem cell, an autologous cell, an allogeneic cell (an allographic cell), and a xenogeneic cell (a xenographic cell) (e.g., porcine beta-islet cell). In some cases, the cell is a host cell for expression of a target. The cell may be a cell for transplant or a transplanted cell. The cell may be a transplanted allographic cell. The cell may be an engineered autographic cell. The cell may be an engineered xenographic cell.

Cell types of interest, include but are not limited to, stem cells, e.g., pluripotent stem cells, hematopoietic stem cells, mesenchymal stem cells, neuronal stem cells, T cells (e.g., CD4⁺T cells, CD8⁺ T cells, cytotoxic T cells, helper T cells, etc.), T regulatory cells, dendritic cells, B Cells, e.g., memory B cells, antigen specific B cells, granulocytes, leukemia cells, lymphoma cells, virus-infected cells (e.g., HIV-infected cells), NK cells, macrophages, monocytes, fibroblasts, epithelial cells, endothelial cells, and erythroid cells.

Target cells of interest include cells that have a convenient cell surface marker or antigen that may be captured by a convenient specific binding member conjugate. In some embodiments, the target cell is selected from HIV-containing cell, a Treg cell, an antigen-specific T-cell populations, tumor cells or hematopoietic progenitor cells (CD34+) from whole blood, bone marrow or cord blood. Any convenient cells having a cell surface protein or cell marker of interest may be targeted for labelling with a cholesterylamine conjugate according to the subject methods. In some embodiments, the target cell includes a cell surface marker selected from a cell receptor and a cell surface antigen. In some cases, the target cell may include a cell surface antigen such as CD11b, CD123, CD14, CD15, CD16, CD19, CD193, CD2, CD25, CD27, CD3, CD335, CD36, CD4, CD43, CD45RO, CD56, CD61, CD7, CD8, CD34, CD1c, CD23, CD304, CD235a, T cell receptor alpha/beta, T cell receptor gamma/delta, CD253, CD95, CD20, CD105, CD117, CD120b, Notch4, Lgr5 (N-Terminal), SSEA-3, TRA-1-60 Antigen, Disialoganglioside GD2 and CD71.

In some cases, the target cell is obtained from a subject. In certain instances, a subject's cells may be removed from the subject, optionally purified, and then labelled according to the subject methods with any convenient cargo moiety. The labelled autologous cells can then be administered back to the subject. As such, in certain instances, the subject methods involve an autologous transplant of a subject's own cells, e.g., stem cells. In certain embodiments, the cells are immune cells which are labelled to include a tumor-targeting agent. Administration of the labelled immune cells back to the subject directs the cells to the tumor to induce a beneficial immune response to the tumor.

In some embodiments, the cell is an autologous cell from a subject and the cell is labelled with a lipid conjugate including an immuno-activating agent (e.g., as described above). In certain instances, the cell is an autologous cell from a patient that is labelled with a first lipid conjugate including an immuno-activating agent (e.g., as described above) and a second lipid conjugate that includes a targeting agent, such as a tumor targeting agent. Such labelled cells may activate the patient's immune system to attack a tumor, and find use in methods of treatment as described herein.

In some embodiments, the cell is a beta-islet cell that is labelled with a targeting agent for the pancreas. Administration of the labelled beta-islet cells to the subject results in directing of the labelled cell to the pancreas and the local production of insulin. In some instances, the beta-islet cells are xenogeneic (xenographic), e.g., porcine beta-islet cells that are administered to a human subject. In some cases, the cells are transplanted from one subject and transferred to another after labelling. In some cases, the cells are autologous. In some cases, the cells are allogeneic.

In some embodiments, the target cell expresses a therapeutic molecule of interest. In certain cases, the cell is selected from a bacteria cell, a yeast cell, an insect cell, a mammalian expression systems (e.g., Chinese hamster ovary cells), and a human derived cell line. A variety of different cell types that can be used as an expression system for a therapeutic molecule of interest can be labelled (e.g., with a targeting agent) and administered to a subject in need thereof. In some cases, such cells find use in the treatment of a lysosomal storage disease, e.g., a genetically inherited disorder that is characterized by a deficiency of a vital enzyme. In patients suffering from a lysosomal storage disease, small increases in the abundance of the missing enzyme can restore their cells (e.g., neurons) to normal function. A cell that expresses a missing enzyme can be administered to a subject in need thereof, where the cell can be labelled with any convenient cargo moiety (e.g., a targeting agent, a cell differentiation agent, etc.) depending on the nature and location of the lysosomal storage disease.

### Methods of Labelling Cells and Use Thereof

Any convenient method may be used to contact the cell with a cholesterylamine conjugate to non-covalently bind the cholesterylamine anchoring group of the conjugate to the membrane of the cells in the sample to produce a labelled cell. An appropriate solution may be used that maintains the biological activity of the components of the cell sample and the lipid conjugate. The solution may be a balanced salt solution, e.g., normal saline, phosphate buffered saline (PBS), Hank's balanced salt solution, etc., conveniently supplemented with fetal calf serum, human platelet lysate or other factors, in conjunction with an acceptable buffer at low concentration, such as from 5-25 mM. Convenient buffers include HEPES, phosphate buffers, lactate buffers, etc. Various media are commercially available and may be used according to the nature of the target analyte, including dMEM, HBSS, dPBS, RPMI, Iscove's medium, etc., in some cases supplemented with fetal calf serum or human platelet lysate. The final components of the solution may be selected depending on the components of the sample which are included.

The temperature at which insertion of the cholesterylamine conjugate into the membrane of the target cell takes place may vary, and in some instances may range from 5° C. to 50° C., such as from 10° C. to 40° C., 15° C. to 40° C., 20° C. to 40° C., e.g., 20° C., 25° C., 30° C., 35° C. or 37° C. In some instances, the temperature at which insertion takes place is selected to be compatible with the biological activity of the cargo moiety and/or the target cell. In certain instances, the temperature is 25° C., 30° C., 35° C. or 37° C. In certain cases, the cargo moiety is an antibody or fragment thereof and the temperature at which insertion takes place is room temperature (e.g., 25° C.), 30° C., 35° C. or 37° C. Any convenient incubation time for insertion may be selected to allow for the persistent display of a desirable amount of cargo moiety at the cell surface, and in some instances, may be 1 minute (min) or more, such as 2 min or more, 10 min or more, 30 min or more, 1 hour or more, 2 hours or more, or even 6 hours or more.

Non-covalent binding and insertion of the cholesterylamine anchoring group of the conjugate into the cell membrane displays the linked cargo moiety (Z) at the cell surface for an extended period of time. As used herein, by extended period of time is meant 1 day or more, such as 2 days or more, 3 days or more, 4 days or more, 5 days or more, 6 days or more, 7 days or more, 8 days or more, 9 days or more, 10 days or more, 2 weeks or more, 3 weeks or more, or even more.

In some instances, the labelled cell provides for a steady state level of display of cargo moiety at the cell surface that is 1% or more by molarity of the total amount of cholesterylamine conjugate taken up by the cell, such as 2% or more, 3% or more, 4% or more, 5% or more, 6% or more, 7% or more, 8% or more, 9% or more, or 10% or more by molarity of the total amount of cholesterylamine conjugate taken up by the cell.

### Methods of Administration

Any of the labelled cells described herein may be administered to a subject of interest. Administration of the subject labelled cells finds use in a variety of applications depending of the cell and the nature of the cargo moiety displayed on the cell surface. In some instances, the labelled cell displays a linked cargo moiety that includes a therapeutic agent and the method is a method of treating a subject for a disease or condition susceptible to treatment by the therapeutic agent. In some embodiments, the method further includes cleaving the linked cargo moiety from the cholesterylamine anchor group to deliver the cargo moiety in situ. In certain cases, the cargo moiety that is released extracellularly is a cell surface displayed therapeutic agent.

In some instances, the method is a method of modulating an immune response in a subject, where the linked cargo moiety displayed at the cell surface is selected from an immuno-inhibiting agent, an immuno-activating agent and an immuno-modulating agent, and administration of the labelled cell modulates an immune response of the subject. In certain embodiments, the linked cargo moiety is an immuno-inhibiting agent; and the cell is a transplanted cell. In certain embodiments, the linked cargo moiety is an immuno-activating agent; and the cell is an autologous cell further comprising a tumor targeting agent. In certain embodiments, the linked cargo moiety is an immuno-modulating agent; and the cell is an immune system cell.

In some instances, the method is a method of targeting a cell in a subject, where the linked cargo moiety Z displayed at the cell surface is a cell adhesion modulating agent; and administration of the labelled cell modulates adhesion of the cell to an extracellular matrix of the subject. In certain cases, modulating adhesion includes inhibiting adhesion of the cell to an extracellular matrix to keep the labelled cell in systemic circulation of the subject for an extended period of time. In certain cases, the adhesion-inhibiting agent is an RGD oligomeric peptide that blocks integrins from binding. In some instances, the cell is a therapeutic agent-expressing cell and systemic circulation of the cell is desirable.

In certain cases, the modulating adhesion includes promoting adhesion of a labelled stem cell to its extracellular matrix to prevent anchorage-dependent cell death.

In some instances, the method is a method of targeting a cell in a subject, where the linked cargo moiety Z displayed at the cell surface is a targeting agent; and administration of the labelled cell targets the cell to particular site in the subject.

In some instances, the method is a method of imaging a site of a subject, where the linked cargo moiety is an imaging agent. Administration of a labelled cell further including a targeting agent can provide for local imaging of a particular site in a subject. For example, a tumor-targeting agent may be used to direct the labelled cell to a tumor site in the subject which can then be imaged, e.g., using positron emission tomography (PET) of a PET emitting probe. In certain instances, the imaging agent is selected from a fluorescent probe, a magnetic resonance imaging agent, an IR dye, a PET emitting probe, and a NMR contrast agent.

Ex vivo and in vitro differentiated cell populations useful as a source of cells may be obtained from any mammalian species, e.g. human, non-human primate, equine, bovine, porcine, canine, feline, murine, etc., particularly human cells. Ex vivo and in vitro differentiated cell populations may include fresh or frozen cells, which may be from a neonate, a juvenile or an adult, and differentiated tissues including skin, muscle, blood, liver, pancreas, lung, intestine, stomach, and other differentiated tissues. Pluripotent cells are optionally deleted from the differentiated cell population prior to labelling and introduction into the recipient. The dose of cells will be determined based on the specific nature of the cell, recipient and nature of condition to be treated, and will generally include from about 10⁶-10¹⁰ cells, which may be provided in suspension, as aggregates, and the like.

To determine the suitability of labelled cell compositions for therapeutic administration, the cells can first be tested in a suitable animal model. At one level, cells are assessed for their ability to survive and maintain their phenotype in vivo. Cell compositions may be administered to immunodeficient animals (such as nude mice, or animals rendered immunodeficient chemically or by irradiation). Tissues are harvested after a period of regrowth, and assessed as to whether the administered cells or progeny thereof are still present. This can be performed by administering cells that express a detectable label (such as green fluorescent protein, or β-galactosidase); that have been prelabeled (for example, with BrdU or [3H] thymidine), or by subsequent detection of a constitutive cell marker (for example, using human-specific antibody). The presence and phenotype of the administered cells can be assessed by immunohistochemistry or enzyme-linked immunosorbent assay (ELISA) using human-specific antibody, or by reverse transcription-polymerase chain reaction (RT-PCR) analysis using primers and hybridization conditions that cause amplification to be specific for human polynucleotides, according to published sequence data.

The subject cells may be labelled and used for tissue reconstitution or regeneration in a human patient or other subject in need of such treatment. The cells are administered in a manner that permits them to graft or migrate to the intended tissue site and reconstitute or regenerate the functionally deficient area.

The subject cells may be administered in any physiologically acceptable excipient. The subject cells may be introduced by injection, catheter, or the like. The subject cells may be frozen at liquid nitrogen temperatures and stored for long periods of time, being capable of use on thawing. If frozen, the subject cells will usually be stored in a 10% dimethylsulfoxide (DMSO), 50% fetal calf serum (FCS), 40% RPMI 1640 medium. Once thawed, the cells may be expanded by use of growth factors and/or feeder cells associated with progenitor cell proliferation and differentiation.

The subject cells can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells, or complementary cell types, especially endothelial cells.

The subject cells may be genetically altered in order to introduce genes useful in the labelled cell, e.g. repair of a genetic defect in an individual, selectable marker, etc. Cells may also be genetically modified to enhance survival, control proliferation, and the like. Cells may be genetically altering by transfection or transduction with a suitable vector, homologous recombination, or other appropriate technique, so that they express a gene of interest. In one embodiment, cells are transfected with genes encoding a telomerase catalytic component (TERT), typically under a heterologous promoter that increases telomerase expression beyond what occurs under the endogenous promoter, (see International Patent Application WO 98/14592). In other embodiments, a selectable marker is introduced, to provide for greater purity of the desired differentiating cell. Cells may be genetically altered using vector containing supernatants over a 8-16 h period, and then exchanged into growth medium for 1-2 days. Genetically altered cells are selected using a drug selection agent such as puromycin, G418, or blasticidin, and then recultured. The subject cells can also be genetically altered in order to enhance their ability to be involved in tissue regeneration, or to deliver a therapeutic agent or gene to a site of administration. A vector is designed using the known encoding sequence for the desired gene, operatively linked to a promoter that is either pan-specific or specifically active in the differentiated cell type.

### Kits

Aspects of the present disclosure include kits for use in practicing the subject methods. The cholesteryl conjugates, precursors thereof, and labelled cells of the present disclosure can be included as components in kits either as starting materials, or provided for use in, for example, the methodologies described above. In some embodiments, the kit includes a cholesterylamine tag or conjugate (e.g., as described herein) having the formula: C-L-Z wherein: C is a cholesterylamine anchor group; L is an optional linker; and Z is a chemoselective reactive group or a linked cargo moiety (e.g., as described herein); and at least one component selected from a cell, a cargo moiety, a positive or negative control (e.g., control cell or reagent such as a non-conjugated cargo moiety), a buffer, etc. The one or more additional components of the kit may be provided in separate containers (e.g., separate tubes, bottles, or wells in a multi-well strip or plate).

In some embodiments, the cholesteryl conjugate precursor includes a chemoselective tag, such as an alkyne tag. In certain cases, the cholesteryl conjugate precursor has the following structure:

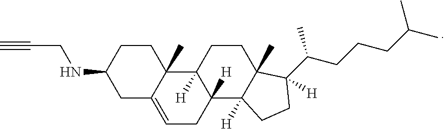

In certain embodiments, the kit further includes reagents for performing a flow cytometric assay. Reagents of interest include, but are not limited to, buffers for reconstitution and dilution, buffers for contacting a cell sample the multichromophore, wash buffers, control cells, control beads, fluorescent beads for flow cytometer calibration and combinations thereof. The kit may also include one or more cell fixing reagents such as paraformaldehyde, glutaraldehyde, methanol, acetone, formalin, or any combinations or buffers thereof. Further, the kit may include a cell permeabilizing reagent, such as methanol, acetone or a detergent (e.g., triton, NP-40, saponin, tween 20, digitonin, leucoperm, or any combinations or buffers thereof. Other protein transport inhibitors, cell fixing reagents and cell permeabilizing reagents familiar to the skilled artisan are within the scope of the subject kits.

The compositions of the kit may be provided in a liquid composition, such as any suitable buffer. Alternatively, the compositions of the kit may be provided in a dry composition (e.g., may be lyophilized), and the kit may optionally include one or more buffers for reconstituting the dry composition. In certain aspects, the kit may include aliquots of the compositions provided in separate containers (e.g., separate tubes, bottles, or wells in a multi-well strip or plate).

In addition, one or more components may be combined into a single container, e.g., a glass or plastic vial, tube or bottle. In certain instances, the kit may further include a container (e.g., such as a box, a bag, an insulated container, a bottle, tube, etc.) in which all of the components (and their separate containers) are present. The kit may further include packaging that is separate from or attached to the kit container and upon which is printed information about the kit, the components of the and/or instructions for use of the kit.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, DVD, portable flash drive, etc., on which the information has been recorded. Yet another means that may be present is a website address which may be used via the Internet to access the information at a removed site. Any convenient means may be present in the kits.

### Utility

The methods and conjugates and labelled cells as described herein may find use in a variety of applications, including diagnostic, therapeutic and research applications, in which the labelling, administration to a subject, detection and/or analysis of a target cell is desirable.

Therapeutic applications of interest include, but are not limited to, cellular applications involving protection of target cells from the immune system, recruitment of the immune system to particular cells or target sites in vivo, exploitation of various cellular adhesion properties, and exploitation of various cell-cell interactions. Specific applications include treatment of tumor in a subject by activating immune cells at the tumor site, and methods of treating a disease condition in a patient by local deliver of a therapeutic agent of interest via a labelled cell.

Diagnostic applications of interest include PET imaging applications, e.g., of a tumor in a subject. Research applications of interest include conjugates for tools for studying cell-cell or cell-extracellular matrix interactions, such as cell adhesion.

Such applications include methodologies such as cytometry, microscopy, immunoassays (e.g. competitive or non-competitive), assessment of receptor bound ligand, flow cytometry, in-situ hybridization, enzyme-linked immunosorbent assays (ELISAs), western blot analysis, magnetic cell separation assays and so forth.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Lipid Anchored Glycopolymers

A panel of lipid anchors were engineered and synthesized to improve the plasma membrane residence times and cell trafficking behavior of glycopolymers. Cholesterylamine (CholA), a lipid that recycles back to the cell surface after internalization, is capable of shuttling glycopolymers through this pathway continuously for up to 10 days. Cholesterylamine anchors were shown to result is persistent display of glycopolymers on the plasma membrane for more than one week after initial cell labeling. Moreover, cells incubated with CholA-anchored glycopolymers internalize some proportion into a storage reservoir that serves as a depot for continuous cell-surface delivery, even within daughter cells derived from a labeled mother cell. Once resident on cells, the CholA glycopolymers replicate known effects of mucins on focal adhesion distribution and cell survival, both in cell culture and in a zebrafish model of tumor cell metastasis.

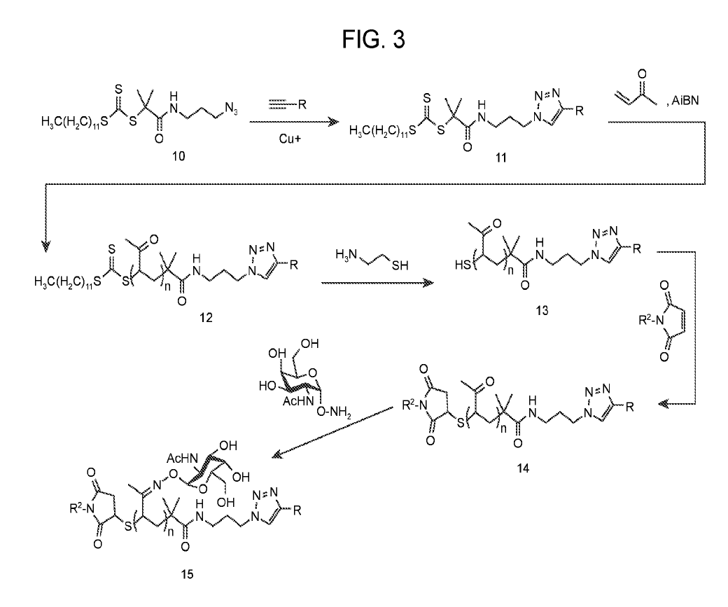

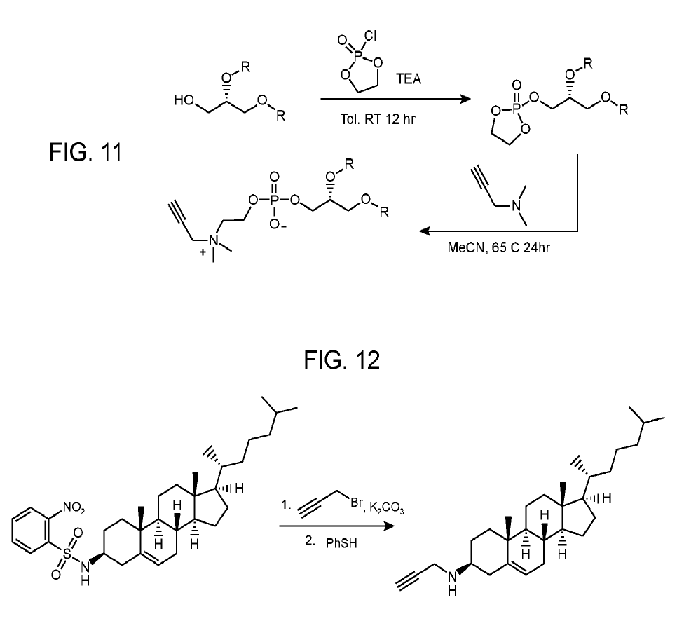

Although the mechanisms underlying native lipid trafficking throughout a cell are not well understood, small changes in lipid structure can have a profound effect on parameters such as internalization kinetics and pathway (See e.g.,. Mukherjee et al., Traffic 2000, 1, 203-211; Maxfield et al., Nat. Rev. Mol. Cell Biol. 2004, 5, 121-β2; Singh et al., J. Biol. Chem. 2006, 281, 30660-30668; Singh et al., J. Cell Biol. 2007, 176, 895-901; and Mayor et al., J. Cell Biol. 1993, 121, 1257-1269). With little basis for rational design, a panel of phosphoglycerolipids was synthesized that varied in length (dimyristoyl DMPC (5) and distearoyl DSPC (6)), regiochemistry (1,3 DMPC (7)), unsaturation (dioleyl, DOPC (8)), or linkage type to glycerol (e.g., diether lipid DEPC (9)) in the hope of improving cell-surface half-life relative to DPPE (FIG. 2). To integrate these lipids into glycopolymers, the synthetic approach outlined in FIG. 3 was crafted. The lipids were outfitted with clickable alkyne groups (see FIG. 11 and the methods of Hajdu et al., J. Org. Chem. 1999, 64, 7727-7737) for conjugation to an azide-functionalized chain-transfer agent (CTA) (10) capable of reversible addition-fragmentation chain-transfer (RAFT) polymerization (Lv et al., J. Colloid Interface Sci. 2011, 356, 16-23). In addition, the two sterol derivatives cholesteryl-succinic acid propargylamide (Chol (3)) and N-propargyl 3β-cholesterylamine (CholA (4)) were prepared using the procedure described in FIG. 12.

After clicking the alkynyl lipids to azide-CTA 10, end-functionalized glycopolymers were generated. Briefly, RAFT polymerization was performed with methyl vinyl ketone (MVK) to generate lipid-anchored poly(MVK) polymers of specified lengths (˜200 DP) and low polydipersity (PDIs=1.1-1.66). The terminal trithiocarbonate was cleaved using cysteamine, and the free thiol was alkylated with a maleimide probe (either Alexa Fluor 488 (AF488) or biotin). Finally, the ketone groups were condensed with aminooxy-N-acetylgalactosamine (GalNAc) to form oxime-conjugated glycopolymers.

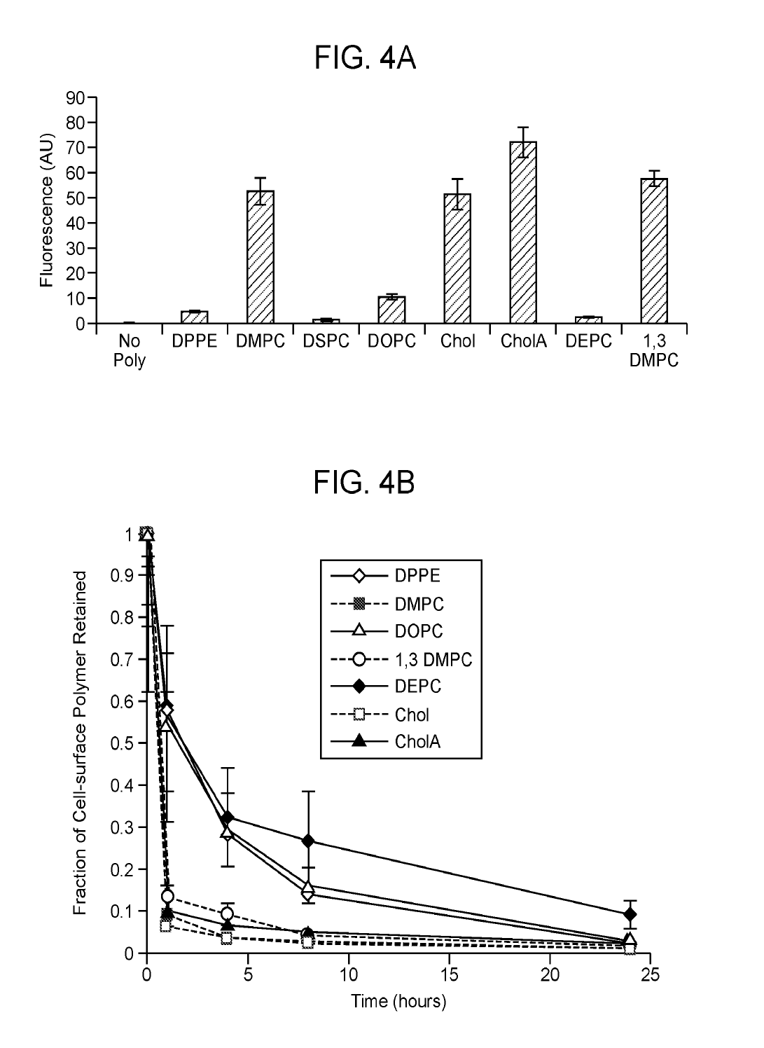

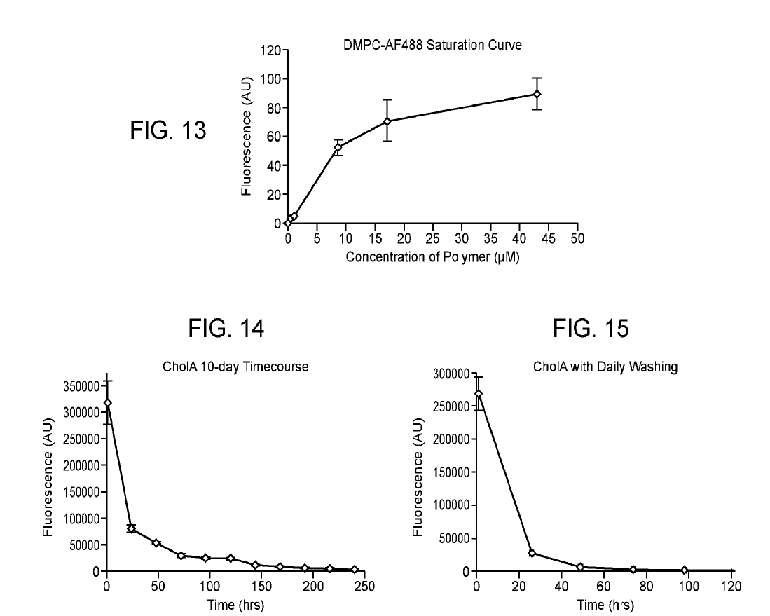

Using AF488 labeled polymers, the efficiency with which each glycopolymer was incorporated into cell membranes was measures. Jurkat cells (10⁷/mL in serum-free media) were incubated for 1 h at room temperature with 10 μM lipid-conjugated glycopolymer, washed, and then measured for fluorescence by flow cytometry (FIG. 4, panel A). For each lipid tested, saturation kinetics were observed, with the amount of glycopolymer displayed on the membrane increasing as a function of loading concentration (see FIG. 13 for DMPC as an example). The polymers with the highest levels of incorporation were the phosphocholine derivatives with shorter fatty acids (DMPC and 1,3 DMPC) and the sterols (Chol and CholA).

To quantitate the persistence of time of glycopolymers on the cell surface, biotin-capped polymers that could be detected with a membrane-impermeant AF488-labeled anti-biotin antibody were used. After incubation with glycopolymers, cells were washed, and incubated in complete media at 37° C. until a given time point, when the cells were washed and cell-surface labeled for flow cytometry analysis. By comparing the mean fluorescence intensity with that obtained immediately after glycopolymer loading, the fraction of polymer remaining on the cell surface could be calculated (FIG. 4, panel B). All of the glycopolymers underwent a significant decrease in cell surface abundance during the first hour after loading.

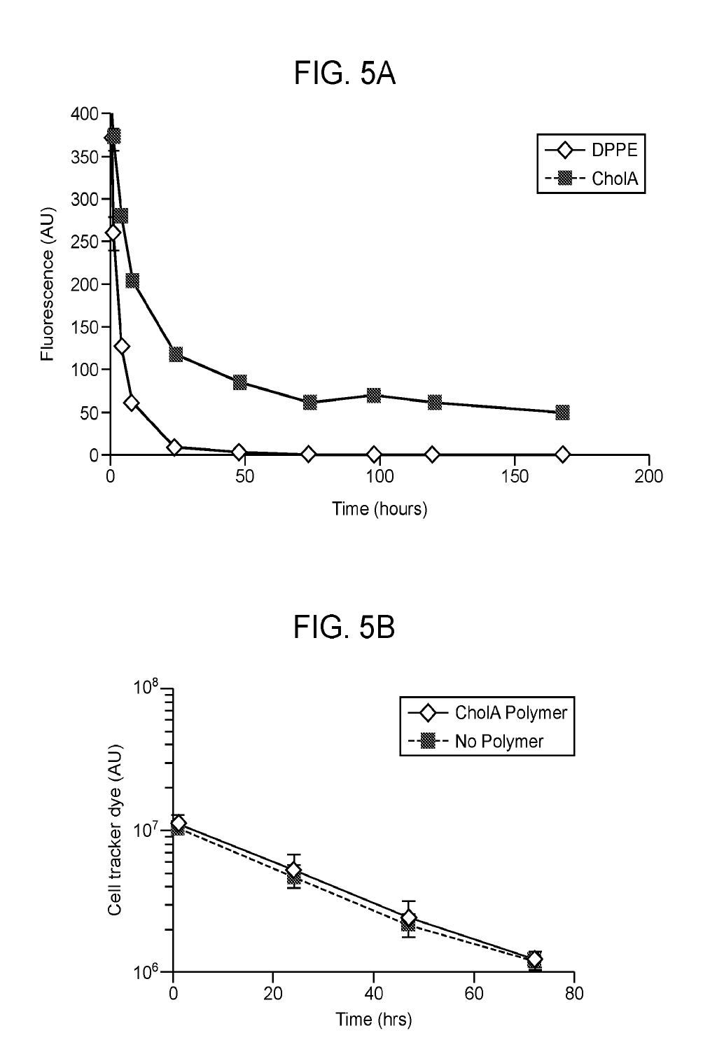

However, analysis of longer time points revealed that CholA-anchored glycopolymers exhibit double exponential kinetics rather than the single exponential function observed for other lipids. After an initial rapid decline, the surface population of CholA-anchored glycopolymers stabilized and remained steady for days, at levels orders of magnitude higher than DPPE (FIG. 5, panel A). The presence of glycopolymers was observed on cell surfaces up to ten days after labeling (FIG. 14), the most persistent synthetic glycoconjugates ever displayed on a cell's surface.

Labeling cells with a cytoplasmic tracking dye along with biotin-capped CholA-anchored glycopolymers allowed monitoring the fate of glycopolymers in a population of dividing cells. While cells show a marked deceleration in their loss of cell-surface polymers after day 1 (FIG. 5, panel A), they actually continue to divide up to day 3 and beyond (FIG. 5, panel B). Importantly, the polymers are passed down uniformly from mother cells to daughter cells as reflected in a stable unimodal distribution of labeling (FIG. 15).

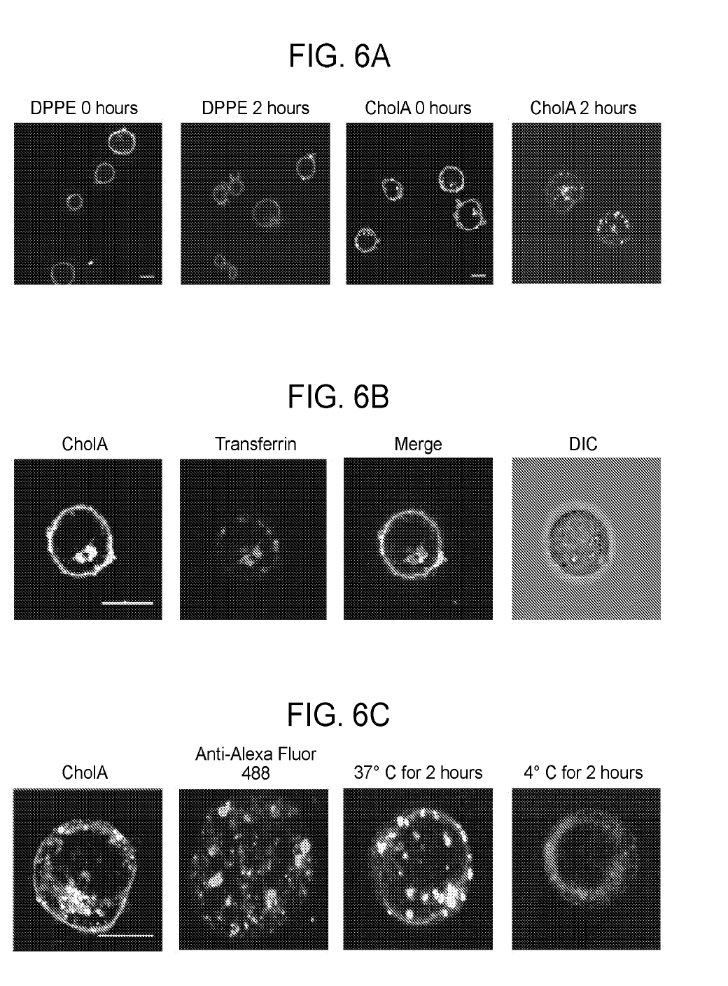

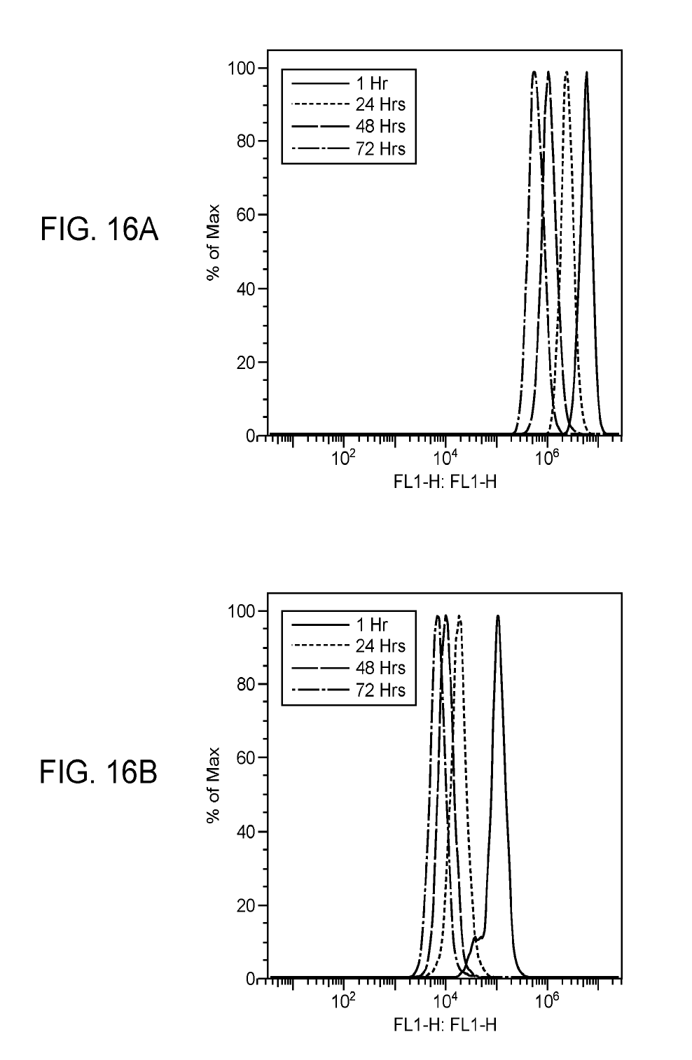

Analysis of the temperature dependence of cell-surface residence time suggested that the rapid initial loss of CholA-anchored glycopolymers is due to endocytosis (FIG. 16). Microscopy analysis of the AF488-capped CholA-anchored polymers revealed that, indeed, the initially rapid internalization yields dense reservoirs of glycopolymer within the cell (FIG. 6, panel A). By contrast, DPPE-anchored glycopolymers formed diffuse internal puncta. It was speculated that CholA was mediating continuous recycling of our glycopolymers from these reservoirs to the cell surface.

The endocytic recycling compartment (ERC) is an organelle responsible for replenishing membrane-associated proteins and lipids once they have been depleted by endocytosis. The plasma protein transferrin is a marker of the ERC. Alexa Fluor 647-labeled transferrin colocalized with the dense reservoirs of CholA-anchored glycopolymers (FIG. 6, panel B). Localization within the ERC, however, does not guarantee recycling. To visualize recycling of the glycopolymer from internal reservoirs back to the cell surface, those AF488-capped glycopolymers present on the cell surface were first quenched with an anti-AF488 antibody (Wang et al., J. Biol. Chem. 1997, 272, 25091-25098.) and observed loss of cell surface labeling as expected (FIG. 6, panel C).

Next, these cells were either warmed for 2 h at 37° C. or kept them on ice, then imaged again. Only the warmed cells were able to replenish their cell surface population of AF488-capped glycopolymers, ostensibly via recycling from internal compartments (FIG. 6, panel C). A model for the mechanism by which CholA-anchored glycopolymers could utilize recycling to replenish the cell surface was obtained, resulting in apparent long-lived cell-surface residence times (FIG. 1).

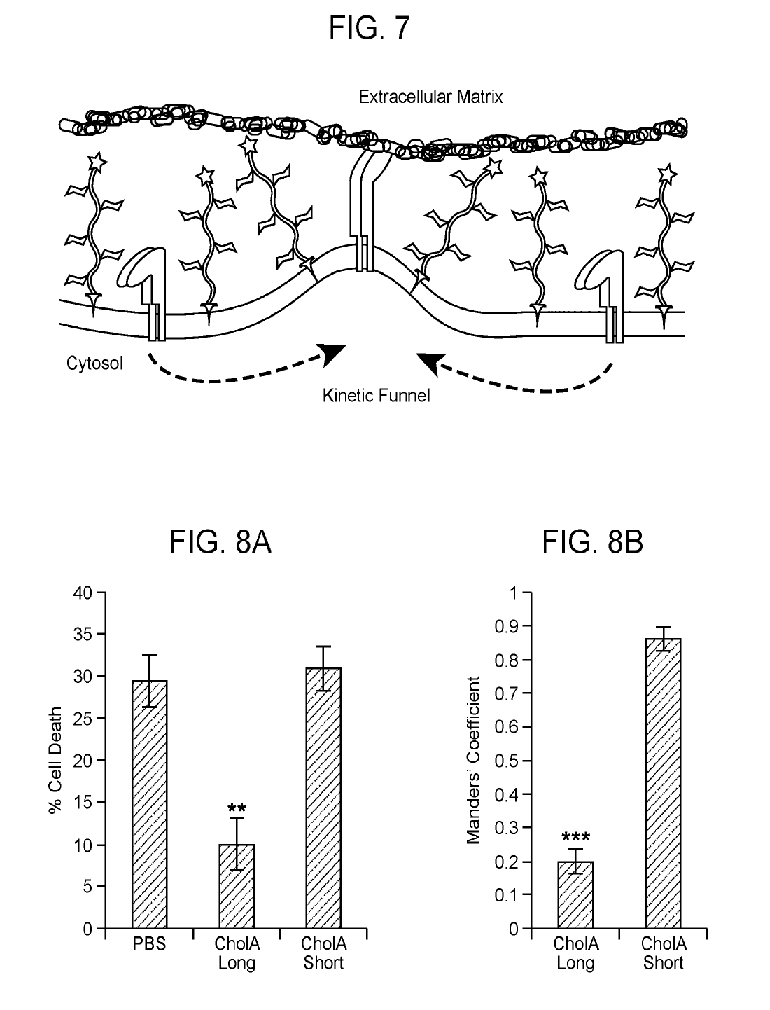

Having identified glycopolymers that stably populate the cell surface for extended periods of time, next it was determined whether they impart biological effects on cells similar to native mucins. Bulky cell-surface mucins such as MUCI can, counterintuitively, promote the adhesion of cells in minimal adhesion settings including environments that mimic the metastatic niche (Paszek et al., Nature 2014, 511, 319-325). By obscuring the majority of binding between integrins and their ligands in the extracellular matrix, a bulky glycocalyx actually drives integrin clustering via a kinetic funnel wherein new bonds are more likely to be made near established ones (FIG. 7) (Paszek et al., PLoS Comput. Biol. 2009, 5, DOI 10.1371/journal.pcbi.1000604). This clustering activates integrins and promotes signaling and cell survival, which can overcome adhesion-mediated programmed cell death (i.e., anoikis) that healthy cells normally undergo.

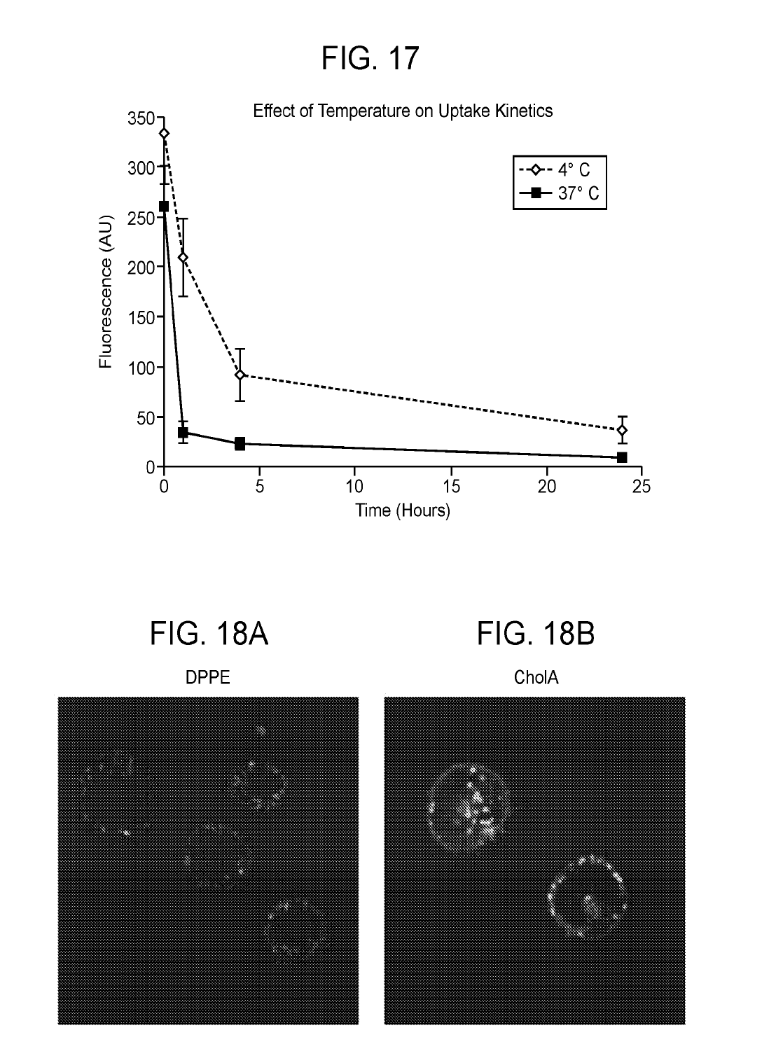

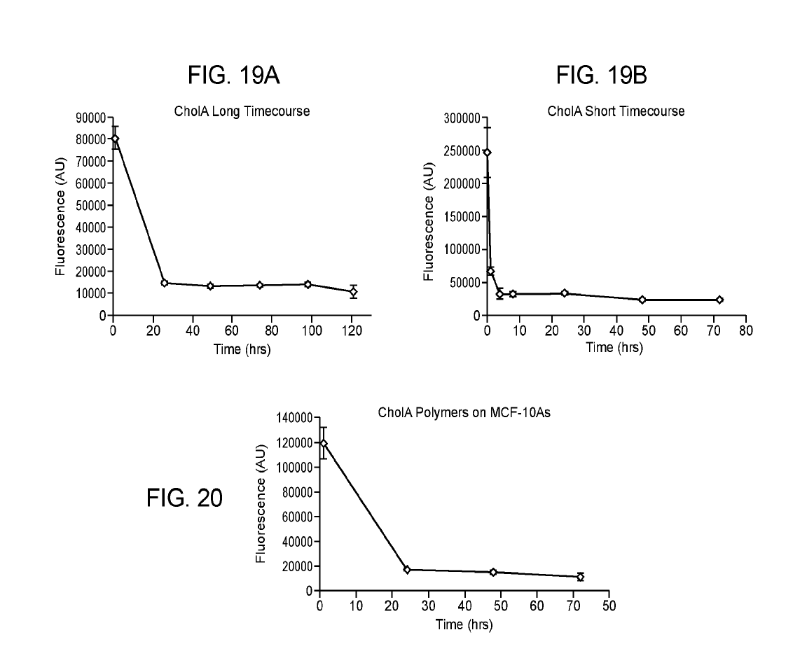

After confirming that the CholA-anchored glycopolymers are themselves nontoxic (FIG. 17), that polymer length does not affect cell-surface persistence (FIG. 18 and FIG. 19), that the CholA-anchored glycopolymers are non-toxic to cells, and that the polymers are long lived on MCF-10A breast epithelial cells (FIG. 20), the polymers were tested for their ability to improve the survival of nonmalignant cells in a model of the minimal adhesion setting. Soft polyacrylamide gels (140 Pa) functionalized with fibronectin offer nonideal substrates for cell growth, and roughly a third of nonmalignant breast epithelial cells (MCF-10As) plated on them die within 24 h (FIG. 8, panel A). CholA-anchored glycopolymers (DP: 719, PDI: 1.1, ˜90 nm in length) much longer than the height of integrins on the cell surface (˜20 nm) prevented more than two thirds of this adhesion-dependent apoptosis. In control experiments, CholA-anchored glycopolymers much shorter than integrins (DP: 36, PDI: 1.20, ˜3 nm) were shown to offer no significant protection from anoikis (FIG. 8, panel A).

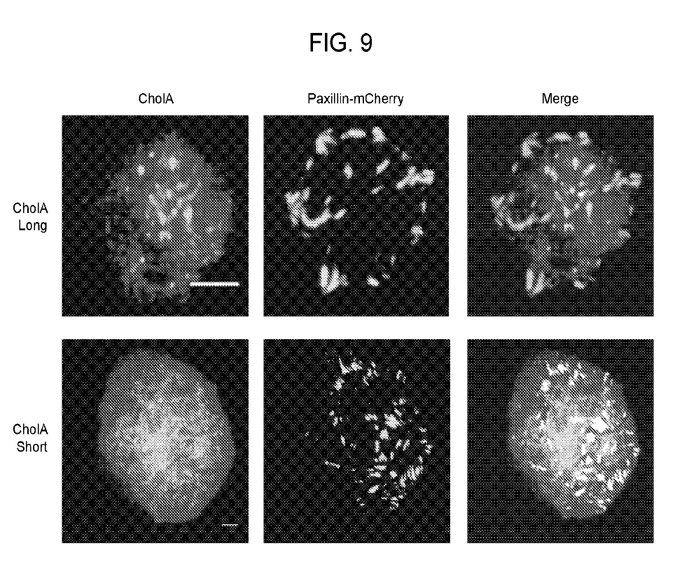

It was hypothesized that increased survival of cells painted with long glycopolymers results from clustering of integrins driven by segregation from glycopolymer rich zones, a phenomenon that is visualized directly by total internal reflection fluorescence (TIRF) microscopy. MCF-10A cells expressing the fluorescent-focal adhesion protein fusion mCherry-paxillin were incubated with biotin-capped CholA-anchored glycopolymers, plated on fibronectin-functionalized coverslips and allowed to adhere, then fixed and stained with AF488-labeled anti-biotin antibody. As shown in FIG. 9, TIRF microscopy analysis revealed that long CholA-anchored glycopolymers are excluded from sites of adhesion formation, while short CholA-anchored glycopolymers are not, consistent with the biophysical model for integrin clustering.

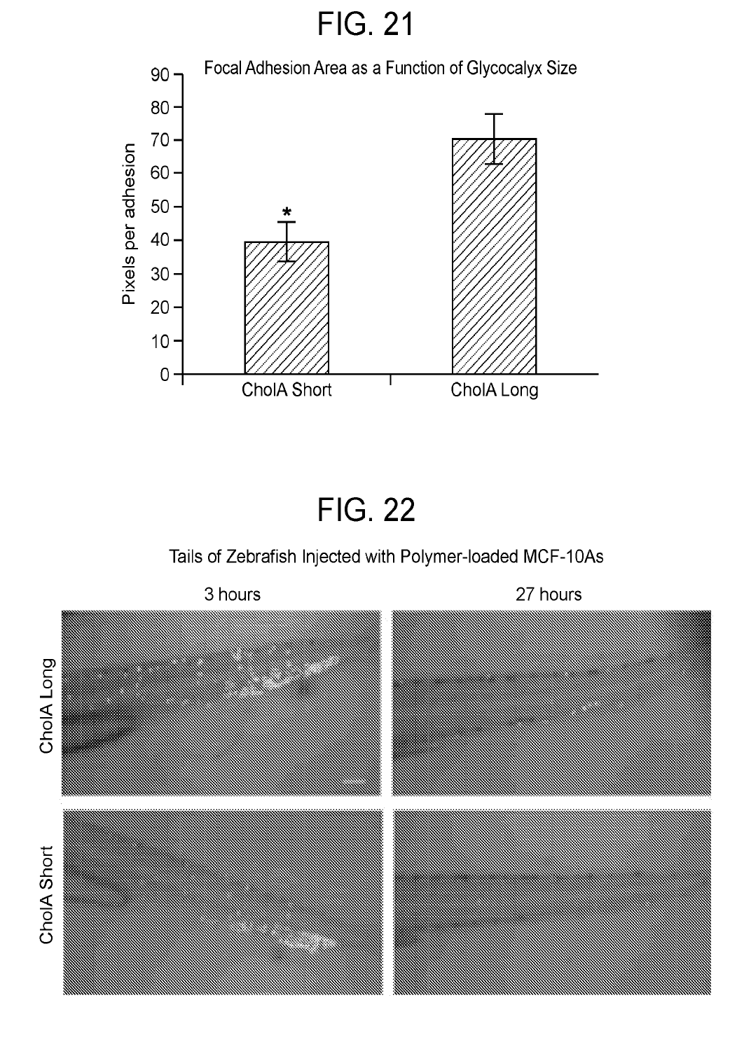

To quantify this effect, the Manders' Colocalization Coefficients (MCC) were calculated for mCherry-paxillin in cells loaded with either long or short glycopolymers. This metric determines the fraction of red pixels that are colocalized with green pixels. In essence, it seeks to answer what percentage of focal adhesion area is also occupied by glycopolymers. With short glycopolymers (3 nm), a high degree of overlap was calculated; 86% of paxillin is coincident with polymer. With long glycopolymers (90 nm), on the other hand, a dramatic segregation was seen; less than 20% of paxillin was coincident with the polymers (FIG. 8, panel B). Furthermore, image analysis showed that the average area per adhesion for cells treated with long CholA-anchored glycopolymers was almost twice that observed on cells treated with short glycopolymers (FIG. 21). These metrics confirm that, like the natural mucin MUC/, the long CholA-anchored glycopolymers are excluded from areas of adhesion and thus likely drive integrin clustering via a kinetic funnel effect, allowing cells to evade anoikis.

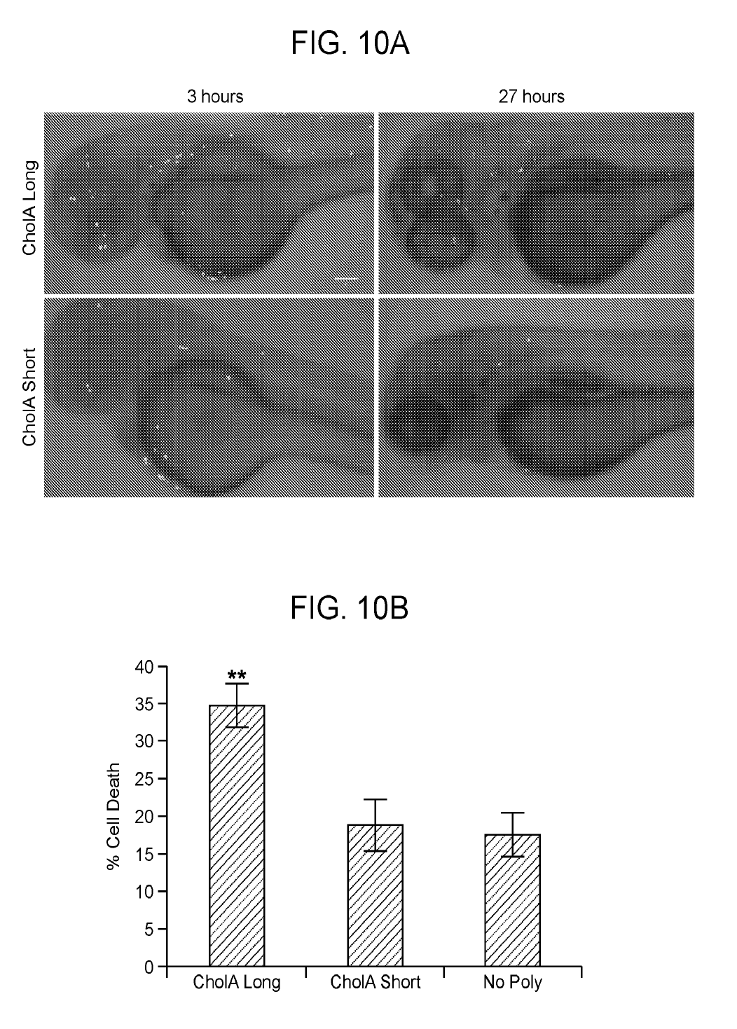

The ability to evade anoikis is a hallmark of malignancy and a prerequisite for tumor metastasis, wherein cells must detach from a primary mass and survive for some time in a non-adherent or suboptimally adherent state. Mucin overexpression may promote these processes by the mechanism outlined above. To determine whether CholA-anchored glycopolymers can similarly promote cell survival and dissemination in vivo, a zebrafish model of metastasis was used (Teng et al., BMC Cancer 2013, 13, 453). Zebrafish embryos 48 hours post-fertilization (hpf) were injected anterior and ventral to the yolk sac, near the vitelline vein and the sinus venosus, with MCF-10A cells constitutively expressing the green fluorescent protein-histone H2B fusion protein (GFP-H2B). MCF-10A cells were first incubated with either long CholA-anchored glycopolymers, short CholA-anchored glycopolymers, or PBS alone. Cells were allowed to disseminate throughout the fish for 3 h, at which point images were acquired and the number of MCF-10A cells present at day 0 was quantified by their fluorescent nuclei. Twenty-four hours later, images were taken again of the same live fish (FIG. 10, panel A and FIG. 22). The MCF-10A cells that were still viable maintained their fluorescent nuclei. To calculate a percent survival, the number of viable cells were counted for each fish and compared to the number from day 0. As in the in vitro model, cells coated with long CholA-anchored glycopolymers showed almost twice the survival rate of cells with short glycopolymers or no polymers at all (FIG. 10b). Thus, CholA-anchored glycopolymers have allowed us to test the effects of a bulky glycocalyx on cell survival over a 27-h period in vivo.

In conclusion, CholA-anchored glycopolymers provide long-lived glycocalyx engineering of naive cells and provide tools to answer questions hereto unapproachable. These reagents can be utilized for a variety of translational applications, such as protection of precious or vulnerable cells (e.g. stem cells) from hostile environments (e.g. the immune system) or tailored homing of engineered cells to target tissues.

FIG. 1: With data from flow cytometry and fluorescence microscopy, a model was developed for the trafficking of CholA-anchored glycopolymers. While constitutively endocytosed, like all lipid-anchored polymers tested, only CholA-anchored glycopolymers appear to recycle from reservoirs inside the cell and replenish cell-surface populations. Scale bars are 10 μm.

FIG. 2: Synthesis of a panel of lipid-anchored glycopolymers. a) Structure and representation of our glycopolymers. A panel of alkynyl lipids was developed that can be clicked to an azide-functionalized RAFT agent (2) and become the anchor for the polymer, R. FIG. 3: Synthesis of dual-end-functionalized glycopolymers. To the known azide-functionalized chain transfer agent (10), an alkynyl lipid (R) is added in either: THF/H₂O with TBTA, CuSO₄ and sodium ascorbate for phosphocholine lipids, or MeOH/CHCl₃ with TBTA, and tetrakis(acetonitrile)copper(I) hexafluorophosphate for alkynyl sterols. The resulting lipid-functionalized CTA (11) is used to polymerize MVK using AIBN as an initiator in 1:1 v/v methyl ethyl ketone at 70° C. to give the polymers (12). The trithiocarbonate is cleaved using cysteamine in DMF. The resulting free-thiol terminated polymers (13) are reacted with 2 equivalents of maleimide-probe. In this report, R² is either biotin or AF488. To the dual functionalized poly-MVK polymer (14), aminooxy GalNAc is added at 1.1 equivalents per ketone in pH 5.5 sodium acetate buffer and acetonitrile, resulting in a probe-capped lipid-anchored glycopolymer (15).

FIG. 4: Glycopolymers with different lipid anchors have different cell-surface kinetics.

Panel A) The efficiency of cell labeling is highly dependent on lipid chemistry. The shortest phosphocholines and the sterol lipids have the highest labeling efficiencies. Jurkat cells were incubated with 10 μM of AF488-capped polymer for 1 h at RT, washed and their fluorescence measured by flow cytometry. Panel B) Relative fractional cell-surface retention of biotin-capped polymers as measured by labeling with an anti-biotin antibody at the given time point and analyzing by flow cytometry. Background, as measured from cells incubated with PBS only, is subtracted and then values are normalized to the measurement taken at time zero for that lipid. None of the lipids in the panel seem to robustly resist uptake by the cell. FIG. 5, panel A: CholA-anchored polymers display double exponential kinetics on long time scales. An initial cell-surface half-life of 0.25 hours is superseded by one of 39 hours. For comparison, DPPE-anchored biotin-capped polymers are nearly undetectable after 24 hours (single-exponential decay kinetics with t_{1/2}=5.8 hours). Panel B) Fluorescence of cells labeled with a cytosolic cell-tracking dye, which can be used to monitor cell division. Cells proliferate for days with or without CholA-anchored polymers. Taken with FIG. 5a, this suggests that the CholA-anchored polymers are transferred to daughter cells. For FIG. 4-FIG. 5, shown are mean minus background+/−standard deviation of three replicate experiments.

FIG. 6: CholA-anchored glycopolymers recycle from reservoirs inside the cell to the cell surface. Panel a) Jurkat cells were labeled with 5 μM AF488-capped glycopolymers for 1 h at RT, then washed and returned to warm complete media to incubate for the indicated times. After 2 hours at 37° C., CholA polymers can be seen accumulating in dense areas within cells, whereas DPPE polymers don't form such reservoirs. Panel B) AF647-labeled human transferrin, a marker for the ERC, colocalizes with the reservoir formed by AF488-capped CholA-anchored glycopolymers, suggesting that recycling contributes to the sustained residence times of the polymer. Panel C) AF488-capped glycopolymers at the cell's surface can be quenched with an anti-AF488 antibody. Cells then allowed to incubate for 2 h display a temperature-dependent return of cell-surface labeling, further suggesting that CholA-anchored polymers are recycling from internal reservoirs to the cell surface.

CholA-anchored glycopolymers are excluded from sites of focal adhesion formation and drive a resistance to anchorage-dependent cell death. FIG. 7 depicts a kinetic funnel model for glycocalyx-driven integrin clustering. By hindering the majority of integrin ligands, bulky ectodomains encourage binding events to occur where bonds already exist, forcing integrins to cluster and thus signal the cell to resist apoptosis. FIG. 8, panel A): Nonmalignant MCF-10A cells plated on soft polyacrylamide substrates undergo anoikis—anchorage-dependent cell death. Long (90 nm) CholA-anchored glycopolymers prevent anoikis, whereas short (3 nm) polymers or vehicle (PBS), do not; thus suggesting a biophysical influence of the glycocalyx on integrin signaling. Error bars are SEM for at least three independent experiments. FIG. 8, panel B): Quantification of the Manders' Colocalization Coefficients for coincidence of mCherry-paxillin with AF488-anti-biotin antibody. Long CholA-anchored glycopolymers show significantly less colocalization than short CholA polymers, suggesting a size-dependent exclusion of polymers from focal adhesions. Scale bars are 5μm. Error bars are SEM for at least five cells quantified from at least two independent experiments. **p<0.01, ***p<0.001.

FIG. 9 shows TIRF imaging of mCherry-paxillin expressing MCF-10A breast epithelial cells incubated with biotin-capped CholA polymers and stained with AF488-anti-biotin. Polymers with large (90 nm) glycodomains are excluded from sites of adhesion, while polymers with short (3 nm) domains are not—evidence for the kinetic trap model of integrin clustering.

FIG. 10 demonstrates long CholA-anchored glycopolymers protect previously nonmalignant cells from adhesion-mediated apoptosis in vivo. Panel A) Zebrafish embryos, 48 hpf, were injected with GFP-H2B expressing MCF-10A cells incubated with either long (90 nm) or short (3 nm) CholA-anchored glycopolymers. Fish were then imaged at 3 hours and again at 27 hours. Cells remaining viable continue to express GFP-H2B, and their nuclei can be quantified. Panel B) Cells with long polymers were almost twice as likely to survive to 27 hours compared to cells with short polymers or cells with no polymers. Scale bars are 100 μm. Error bars are SEM of five biological replicates. **p<0.01.

FIG. 11. General synthesis of alkynyl phosphocholines. A synthetic strategy using nucleophilic ring-opening of a cyclic phosphate triester to yield the desired zwitterionic phosphocholine derivative. Diacyl or diether glycerol is combined with 2-chloro-1,3,2-dioxaphospholane 2-oxide and triethylamine in toluene and stirred for 12 hrs at room temperature. To the crude resulting cyclic phosphate triester is added N,N-dimethylpropargylamine in anhydrous acetonitrile, and let stir at 65° C. for 24 hours.

FIG. 12. Synthesis of alkynyl cholesterylamine To the known N-cholesteryl-2-nitrobenzenesulfonamide, propargyl bromide and potassium carbonate were added in anhydrous THF and let stir overnight at 85° C. To the filtered and dried crude product, thiophenol was added in acetonitrile and let stir at room temperature overnight. Flash chromatography gave the pure product in 85% yield over two steps.

FIG. 13. Lipid-anchored glycopolymers exhibit saturation on cell surfaces. Jurkat cells (10⁷/mL) were incubated with AF488-polymers at the given concentration or PBS in serum free media for 1 hr at RT. The cells were then washed, and their fluorescence measured by flow cytometry. Shown are mean and standard deviation of three replicate experiments minus background as measured by cells incubated with PBS instead of polymer.

FIG. 14. CholA polymers can be measured on cell surfaces as long as ten days after labeling. Jurkat cells (10⁷/mL) were incubated with biotin-polymers at 10 μM or PBS alone in serum free media for 1 hr at RT. The cells were then washed and added to complete media and incubated at 37° C. for the time indicated. At the times indicated, cells were removed, washed with PBS and incubated with AF488-anti-biotin (1:500) for 20 min on ice in 1% FBS in PBS, washed again, and then their fluorescence measured by flow cytometry. Shown are the mean and standard deviation of three replicate experiments minus background as measured by cells incubated with PBS instead of polymer.

FIG. 15. CholA polymers can be driven by mass action out of the cell surface. Jurkat cells (10⁷/mL) were incubated with biotin-capped CholA-polymers at 10 μM or PBS alone in serum free media for 1 hr at RT. The cells were then washed and added to complete media and incubated at 37° C. for the time indicated. Every 24 hours the cells were pelleted, washed with PBS and resuspended in warm complete media. At the times indicated, cells were removed, washed with PBS and incubated with AF488-anti-biotin (1:500) for 20 min on ice in 1% FBS in PBS, washed again, and then their fluorescence measured by flow cytometry. Polymers could be measured up to day 4. Shown are the mean and standard deviation of three replicate experiments minus background as measured by cells incubated with PBS instead of polymer.

FIG. 16. CholA polymers are distributed equitably between daughter cells. Jurkat cells (10⁷/mL) were incubated with biotin-polymers at 10 μM and CellTracker green (25 μM) in serum free media for 1 hr at RT. The cells were then washed and added to complete media and incubated at 37° C. for the time indicated. At the times indicated, cells were removed, washed with PBS and incubated with AF647-anti-biotin (1:500) for 20 min on ice in 1% FBS in PBS, washed again, and then their fluorescence measured by flow cytometry (FL1 channel measures the CellTracker Green and FL4 the AF647 anti-biotin). Shown are the histograms of at least 104 cells for each time point. CellTracker green roughly halves every 24 hrs (see FIG. 3 D). Anti-biotin labeling is unimodal even after three generations of cell division showing that daughter cells don't form divergent populations with respect to polymer presentation.

FIG. 17. Uptake of CholA polymers is temperature dependent. Jurkat cells (10⁷/mL) were incubated with biotin-polymers at 1 μM or PBS alone in serum free media for 1 hr at RT. The cells were then washed and added to complete media and incubated at either 4 or 37° C. for the time indicated. At the times indicated, cells were removed, washed with PBS and incubated with AF488-anti-biotin (1:500) for 20 min on ice in 1% FBS in PBS, washed again, and then their fluorescence measured by flow cytometry. Shown are mean and standard deviation of three replicate experiments minus background as measured by cells incubated with PBS instead of polymer.

FIG. 18. CholA-anchored glycopolymers are still present on cell surfaces after 24 hours. Jurkat cells (10⁷/mL) were incubated with AF488-capped polymers (5 μM) in serum free media for 1 hr at RT. The cells were then washed and added to complete media and incubated at 37° C. for 24 hours. The cells were pelleted, washed with PBS, and then imaged by confocal microscopy.

FIG. 19, top panel. Long CholA polymers also exhibit long lifetimes. Jurkat cells (10⁷/mL) were incubated with biotin-polymers (90 nm, DP =719) at 10 μM or PBS in serum free media for 1 hr at RT. The cells were then washed and added to complete media and incubated at 37° C. for the time indicated. At the times indicated, cells were removed, washed with PBS and incubated with AF488-anti-biotin (1:500) for 20 min on ice in 1% FBS in PBS, washed, and then their fluorescence measured by flow cytometry. Shown are the mean and standard deviation of three replicate experiments minus background as measured by cells incubated with PBS instead of polymer.

FIG. 19, bottom panel. Short CholA polymers also exhibit long lifetimes. Jurkat cells (10⁷/mL) were incubated with biotin-polymers (3 nm, DP=36) at 10 μM or PBS in serum free media for 1 hr at RT, according to the method described above.

FIG. 20 shows that CholA conjugates are long-lived on MCF-10As. MCF-10A cells (10⁷/mL) were incubated with biotin-polymers at 10 μM or PBS in serum free media for 1 hr at RT. The cells were then washed, added to complete media and allowed to adhere, then incubated at 37° C. for the time indicated. At the times indicated, cells were trypsinized, washed with PBS and incubated with AF488-anti-biotin (1:500) for 20 min on ice in 1% FBS in PBS, washed again, and then their fluorescence measured by flow cytometry. Shown are the mean and standard deviation of three replicate experiments minus background as measured by cells incubated with PBS instead of polymer.

FIG. 21. Long CholA-anchored polymers increase the adhesion size of MCF-10As. mCherry-paxillin expressing MCF-10A cells (10⁷/mL) were incubated with biotin-polymers at 10 μM or PBS in serum free media for 1 hr at RT. The cells were then washed, added to complete media and allowed to adhere to fibronectin-functionalized coverslips, and incubated at 37° C. for two hours. Cells were washed briefly, then fixed with 4% formaldehyde in PBS for 15 min at RT, and then rinsed three times with PBS. AF488-antibiotin antibody (1:200) in 0.5% BSA in PBS was added on ice for 20 min, then rinsed three times. The coverslips were then imaged using TIRF. Focal adhesion areas were calculated using ImageJ. Shown is the average of adhesions from at least 5 cells from at least two independent experiments+/−SEM. *p<0.05

FIG. 22. Tails of zebrafish injected with GFP-H2B expressing MCF-10A breast epithelial cells loaded with either long or short CholA-anchored polymers, then imaged at 3 and then 27 hours. Scale bar is 100 μm.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

Notwithstanding the appended claims, the disclosure set forth herein is also described by the following clauses.

1. A method of labelling a cell, the method comprising:

contacting the cell with a cholesteryl-cargo conjugate having the formula:

C-L-Z (I)

wherein:
C is a cholesterylamine anchor group;
L is an optional linker; and
Z is a linked cargo moiety; to non-covalently bind the cholesterylamine anchoring group to the cell membrane thereby displaying Z at the cell surface for an extended period of time.

2. The method of clause 1, wherein the extended period of time is 1 day or more.

3. The method of any one of the preceding clauses, wherein 2% or more by molarity of the cargo moiety taken up by the cell is displayed at the cell surface.

4. The method of any one of the preceding clauses, wherein the cholesteryl-cargo conjugate is recycled to and from the cell surface via internalized vesicles that non-covalently bind the cholesterylamine anchor group.

5. The method of any one of the preceding clauses, wherein the linked cargo moiety is selected from an immunoinhibiting agent, an immunoactivating agent, an immunomodulating agent, an adhesion modulating agent, a cell surface displayed therapeutic agent, a targeting agent, a cell differentiation agent and an imaging agent.

6. The method of any one of the preceding clauses, further comprising cleaving the linked cargo moiety from the cholesterylamine anchor group.

7. The method of any one of the preceding clauses, wherein the cell is selected from an immune cell, a stem cell, an autologous cell, an allographic cell, xenographic cell and a host cell for expression of a target.

8. The method of any one of the preceding clauses, further comprising administering the labelled cell to a subject.

9. The method of any one of the preceding clauses, wherein the linked cargo moiety is a cell surface displayed therapeutic agent.

10. The method of any one of the preceding clauses, wherein the linked cargo moiety is an imaging agent selected from a fluorescent probe, an IR dye, a PET emitting probe and a NMR contrast agent.

11. The method of any one of the preceding clauses, wherein the linked cargo moiety is a cell differentiation agent.

12. The method of any one of the preceding clauses, wherein the wherein the linked cargo moiety is selected from a glycan, a glycopolymer, a protein, a small molecule and a peptide.

13. The method of any one of the preceding clauses, wherein the conjugate has the structure:

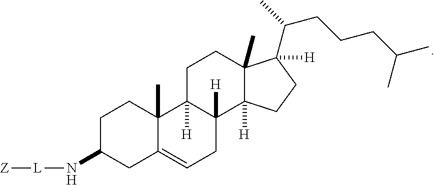

14. The method of anyone of the preceding clauses, wherein Z has the following structure:

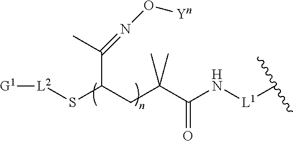

wherein:

each Y is independently a glycan or sidechain group
L¹ and L² are each optional linkers;
n is an integer from 1 to 10,000; and
G¹ is selected from H, an alkyl, a substituted alkyl and a detectable moiety.

15. The method of clause 14, wherein Z has the following structure:

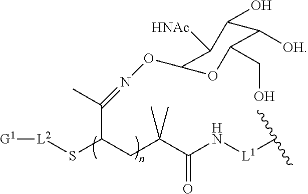

16. A method of modulating an immune response in a subject, the method comprising: administering to a subject a labelled cell comprising:

a cell; and
a cholesteryl-cargo conjugate having the formula:

C-L-Z (I)
wherein:
C is a cholesterylamine anchor group bound to the cell membrane;
L is an optional linker; and
Z is a linked cargo moiety displayed at the cell surface and selected from an immuno-inhibiting agent, an immuno-activating agent and an immuno-modulating agent;
   to modulate an immune response of the subject.

17. The method of clause 16, wherein:

the linked cargo moiety is an immuno-inhibiting agent; and
the cell is a transplanted cell.

18. The method of clause 16, wherein: the linked cargo moiety is an immuno-activating agent; and the cell is an autologous cell further comprising a tumor targeting agent.

19. The method of clause 16, wherein: the linked cargo moiety is an immuno-modulating agent; and the cell is an immune system cell.

20. A method, comprising:

administering to a subject a labelled cell comprising:

a cell; and

a cholesteryl-cargo conjugate having the formula:

C-L-Z (I)
wherein:
C is a cholesterylamine anchor group bound to the cell membrane;
L is an optional linker; and
Z is a linked cargo moiety displayed at the cell surface and is a cell adhesion modulating agent or a targeting agent;

to locally target the cell or to modulate adhesion of the cell to an extracellular matrix of the subject.

21. The method of clause 20, wherein: the linked cargo moiety is an adhesion-promoting agent; and the cell is a stem cell.

22. The method of clause 20, wherein: the linked cargo moiety is an adhesion-inhibiting agent; and the cell is a therapeutic agent expressing cell.

23. The method of clause 20, wherein the linked cargo moiety is a targeting agent.

24. A cholesterylamine conjugate having the formula:

C-L-Z (I)

wherein:

C is an amine-linked cholesterylamine;

L is an optional linker; and

Z is selected from an immunoinhibiting agent, an immunoactivating agent, an immunomodulating agent, an adhesion modulating agent, a cell surface displayed therapeutic agent, a targeting agent, a cell differentiation agent and an imaging agent.

25. The cholesterylamine conjugate of clause 24, wherein Z comprises a glycan selected from Sia, N-acetylneuraminic acid; lactose, galactose-b1,4-glucose; GalNAc, N-acetylgalactosamine; GD3, N-acetylneuraminic acid-α2,8-N-acetylneuraminic acid-α2,3-galactose-β1,4-glucose; and SiaLex, N-acetylneuraminic acid-α2,3-galactose-β1,4-(α1,3-fucose)-N-acetylglucosamine

26. The cholesterylamine conjugate of any one of clauses 24 and 25, wherein Z has the following structure:

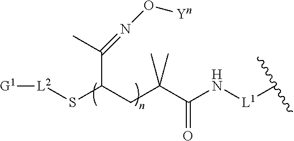

wherein:

each Y is independently a glycan or sidechain group

L¹ and L² are each optional linkers;

n is an integer from 1 to 10,000; and

G¹ is selected from H, an alkyl, a substituted alkyl and a detectable moiety.

27. The cholesterylamine conjugate of any one of clauses 24-26, wherein Z is a glycopolymeric mimic of a mucin.

28. The cholesterylamine conjugate of any one of clauses 24-26, wherein the cholesterylamine conjugate has the structure:

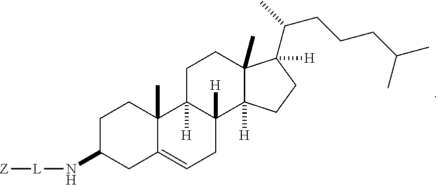

29. The cholesterylamine conjugate of any one of clauses 24-28, wherein Z has the following structure:

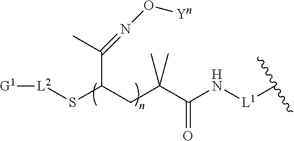

wherein:
each Y is independently a glycan or sidechain group
L¹ and L² are each optional linkers;
n is an integer from 1 to 10,000; and
G¹ is selected from H, an alkyl, a substituted alkyl and a detectable moiety.

30. The cholesterylamine conjugate of clause 29, wherein Z has the following structure:

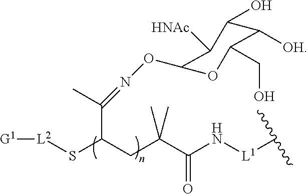

31. A labelled cell, comprising:

a cell; and
a cholesterylamine-cargo conjugate having the formula:

C-L-Z (I)

wherein:

C is a cholesterylamine anchor group bound to the cell membrane;

L is an optional linker; and

Z is a linked cargo moiety displayed at the cell surface, wherein Z is selected from an immunoinhibiting agent, an immunoactivating agent, an immunomodulating agent, an adhesion modulating agent, a cell surface displayed therapeutic agent, a targeting agent, a cell differentiation agent and an imaging agent.

32. The labelled cell of clause 31, wherein the cargo moiety is a glycopolymer.

33. The labelled cell of any one of clauses 31 to 32, wherein the cell comprises a plurality of cholesterylamine-cargo conjugates displaying a plurality of linked cargo moieties at the cell surface.

34. The labelled cell of clause 33 wherein the plurality of linked cargo moieties defines a glycocalyx layer at the surface of the labelled cell.

35. The labelled cell of any one of clauses 31 to 34, wherein the cell is selected from a stem cell, a transplanted allographic cell, an autographic cell, a xenographic cell, a bacterial cell and an immune cell.

36. The labelled cell of any one of clauses 31 to 35, further comprising a second cholesterylamine-cargo conjugate comprising a second distinct linked cargo moiety that is located at the cell surface.

37. The labelled cell of any one of clauses 31 to 36, wherein the cholesteryl-anchored tag has the structure:

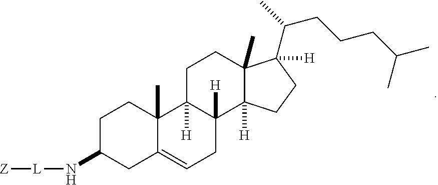

38. The labelled cell of clause 31, wherein Z is a glycopolymeric mimic of a mucin.

39. The labelled cell of any one of clauses 31 to 38, wherein Z has the following structure:

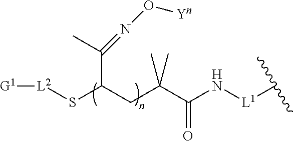

wherein:
each Y is independently a glycan or a sidechain group;
L¹ and L² are each optional linkers;
n is an integer from 1 to 10,000; and
G¹ is selected from H, an alkyl, a substituted alkyl and a detectable moiety.

40. The labelled cell of clause 39, wherein each Y is selected from Sia, N-acetylneuraminic acid; lactose, galactose-b1,4-glucose; GalNAc, N-acetylgalactosamine; GD3, N-acetylneuraminic acid-α2,8-N-acetylneuraminic acid-α2,3-galactose-β1,4-glucose; SiaLex, N-acetylneuraminic acid-α2,3-galactose-β1,4-(α1,3 -fucose)-N-acetylglucos amine, acetic acid and glycerol.

41. The labelled cell of clause 39, wherein Z has the following structure:

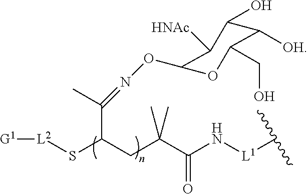

42. A kit for labelling a cell, the kit comprising:

a cholesterylamine conjugate or precursor thereof having the formula:

C-L-Z (I)

wherein:

C is a cholesterylamine anchor group;

L is an optional linker; and

Z is a chemoselective reactive group or a linked cargo moiety; and at least one component selected from a cell, a cargo moiety, and a positive or negative control.

43. The kit of clause 42, wherein the cholesterylamine conjugate or precursor thereof is described by any one of clauses 24-30.

What is claimed is:

1. A method of labelling a cell, the method comprising:
contacting the cell with a cholesteryl-cargo conjugate having the formula:
C-L-Z (I)
wherein:
C is a cholesterylamine anchor group;
L is an optional linker; and
Z is a linked cargo moiety;
to non-covalently bind the cholesterylamine anchoring group to the cell membrane thereby displaying Z at the cell surface for an extended period of time.

2. The method of claim 1, wherein the extended period of time is 1 day or more.

3. The method of claim 1, wherein 2% or more by molarity of the linked cargo moiety taken up by the cell is displayed at the cell surface.

4. The method of claim 1, wherein the cholesteryl-cargo conjugate is recycled to and from the cell surface via internalized vesicles that non-covalently bind the cholesterylamine anchor group.

5. The method of claim 1, wherein the linked cargo moiety is selected from an immunoinhibiting agent, an immunoactivating agent, an immunomodulating agent, an adhesion modulating agent, a cell surface displayed therapeutic agent, a targeting agent, a cell differentiation agent and an imaging agent.

6. The method of claim 1, further comprising cleaving the linked cargo moiety from the cholesterylamine anchor group.

7. The method of claim 1, wherein the wherein the linked cargo moiety is selected from a glycan, a glycopolymer, a protein, a small molecule and a peptide.

8. The method of claim 1, wherein the cholesterylamine conjugate has the structure:

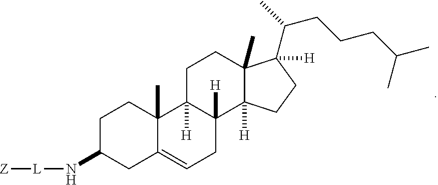

9. The method of claim 1, wherein Z has the following structure: 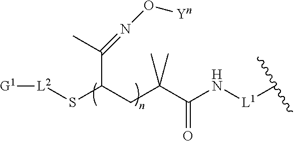
wherein:
each Y is independently a glycan or sidechain group
L¹ and L² are each optional linkers;
n is an integer from 1 to 10,000; and
G¹ is selected from H, an alkyl, a substituted alkyl and a detectable moiety.

10. The method of claim 9, wherein Z has the following structure:

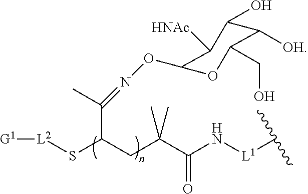

11. A method of modulating an immune response in a subject, the method comprising:
administering to a subject a labelled cell comprising:
a cell; and
a cholesteryl-cargo conjugate having the formula:
C-L-Z (I)
wherein:
C is a cholesterylamine anchor group bound to the cell membrane;
L is an optional linker; and
Z is a linked cargo moiety displayed at the cell surface and selected from an immuno-inhibiting agent, an immuno-activating agent and an immuno-modulating agent;
to modulate an immune response of the subject.

12. The method of claim 11, wherein:
the linked cargo moiety is an immuno-inhibiting agent; and
the cell is a transplanted cell.

13. The method of claim 11, wherein:
the linked cargo moiety is an immuno-activating agent; and
the cell is an autologous cell further comprising a tumor targeting agent.

14. The method of claim 11, wherein:
the linked cargo moiety is an immuno-modulating agent; and
the cell is an immune system cell.

15. A cholesterylamine conjugate having the formula:
C-L-Z (I)
wherein:
C is an amine-linked cholesterylamine;
L is an optional linker; and
Z is selected from an immunoinhibiting agent, an immunoactivating agent, an immunomodulating agent, an adhesion modulating agent, a cell surface displayed therapeutic agent, a targeting agent, a cell differentiation agent and an imaging agent.

16. The cholesterylamine conjugate of claim 15, wherein Z comprises a glycan selected from Sia, N-acetylneuraminic acid; lactose, galactose-b1,4-glucose; GalNAc, N-acetylgalactosamine; GD3, N-acetylneuraminic acid-α2,8-N-acetylneuraminic acid-α2,3-galactose-β1,4-glucose; and SiaLex, N-acetylneuraminic acid-α2,3-galactose-β1,4-(α1,3-fucose)-N-acetylglucosamine

17. The cholesterylamine conjugate of claim 15, wherein Z is a glycopolymeric mimic of a mucin.

18. The cholesterylamine conjugate of claim 15, wherein the cholesterylamine conjugate has the structure:

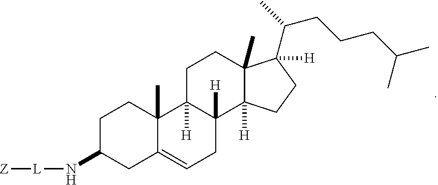

19. The cholesterylamine conjugate of claim 15, wherein Z has the following structure: 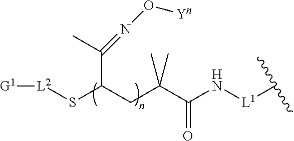
wherein:
each Y is independently a glycan or sidechain group
L¹ and L² are each optional linkers;
n is an integer from 1 to 10,000; and
G¹ is selected from H, an alkyl, a substituted alkyl and a detectable moiety.

20. The cholesterylamine conjugate of claim 19, wherein Z has the following structure: [chemical structure]
